(19) United States
(12) Patent Application Publication
(10) Pub. No.: US 2009/0082644 A1
(43) Pub. Date: Mar. 26, 2009

(54) Devices, Systems, Kits and Methods for Treatment of Obesity

(76) Inventor: Jiayi Li, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 12/047,600

(22) Filed: Mar. 13, 2008

Related U.S. Application Data
(60) Provisional application No. 60/895,006, filed on Mar. 15, 2007.

(51) Int. Cl. A61F 2/02; A61B 5/07

(52) U.S. Cl. 600/302; 606/192

(57) ABSTRACT

Methods, systems and kits are disclosed for devices suitable for use in the treatment of obesity in patients, either human or animal. The devices comprise an inflatable balloon having a proximal end and a distal end, the inflatable balloon further comprising an interior chamber adapted and configured to receive a filling material; a port in communication with the interior chamber of the inflatable balloon. The inflatable balloon can further be adapted and configured to achieve a deployment shape at least partially conformable to an interior dimensional shape of a stomach of the patient and further wherein the inflatable balloon is adapted and configured to have a distal diameter less than or equal to a proximal diameter.

### CROSS-REFERENCE

This application claims the benefit of U.S. Provisional Application No. 60/895,006, filed Mar. 15, 2007, which application is incorporated herein by reference.

### BACKGROUND OF THE INVENTION

It is currently estimated that more than 50 million Americans are overweight; 40 million are obese; and anywhere from 3 million to 9 million are morbidly obese. Obesity is a concern that is not unique to the United States. In Asia, China's Ministry of Health found that the number of obese Chinese had doubled to 60 million between 1992 and 2004. There are an estimated 500 million overweight and 250 million obese people in the world. The World Health Organization (WHO) and American Centers for Disease Control (CDC) consider persons with a body mass index (“BMI”) greater than 25 overweight, a BMI greater than 30 obese, and a BMI greater than 40 morbidly obese. BMI is expressed, for example, as weight/height² (kg/m²).

Obesity is associated with many diseases including, for example, diabetes, cardiovascular disease, hypertension, stroke, dyslipidemia, sleep apnea, some forms of cancer (e.g., uterine, breast, colorectal, kidney, and gallbladder), and osteoarthritis. Many disease states and health risks can be significantly improved by weight loss. It is estimated that 80% of Type II Diabetes is related to obesity, while 70% of cardiovascular disease is related to obesity. The health impact from being overweight or obese has been estimated to cost $117 billion in the United States, with an estimated direct cost of $61 billion and indirect cost of $56 billion. See, e.g., Finkelstein, E. A. et al. “National Medical Expenditures Attributable to Overweight and Obesity: How Much and Who's Paying?” Health Affairs (Web Exclusive): W3-219-W3-226 (May 14, 2003).

Many people spend years trying to lose weight to achieve a normal weight with little success. Diets range the gamut of starvation, to formula, to high protein, low protein, high fat, low fat, etc.

Various surgical approaches to weight loss have also been used or contemplated over the years. Adjustable gastric banding is a bariatric operation where a small pouch is created in the upper part of the stomach by wrapping an adjustable band around the stomach about 20 mm below the gastro-esophageal junction. The band leaves only a narrow passageway (stoma) from the newly created pouch into the larger lower section of the stomach. The reduced capacity of the pouch that is created results in patient's experiencing a rapid sense of fullness.

Stomach or gastric, bypass surgery is a malabsorptive bariatric surgical treatment or approach that involves two basic procedures. First, the size of the stomach is reduced (e.g., by partial removal of the stomach, using gastric staples or a gastric silastic ring). Second, the bypass surgery alters the anatomy of the digestive tract, so that food bypasses a first section of the small intestine, thus reducing the amount of calories (and nutrition) which can be absorbed. Some bypass surgeries (such as biliopancreatic diversion) bypass the duodenum and jejunum completely and connect the stomach directly to the ileum, the final section of the small intestine. Other operations (like Roux-en-Y) bypass less of the intestine thus permitting more calories and nutrients to be absorbed. These interventions have significant complications and several undesirable side-effects, including a death rate of approximately 1 in 50 patients.

Yet another alternative procedure employs deploying space-occupying structures into the stomach, often referred to as “gastric balloons.” Gastric balloons, or intragastric devices, may be introduced through the esophagus and inflated in situ in order to occupy a significant volume within the stomach. The use of conventional gastric balloons has presented a number of problems. For example, in the event of a sudden or slow deflation of the gastric balloon, it is possible for the balloon to pass through the pylorus in the stomach and enter the intestine. Such unintentional passage of the deflated balloon into the intestine can result in intestinal obstruction which can be life-threatening. The risk of deflation of the gastric balloon is further exacerbated by the fact that the patient may not immediately be aware that the balloon has deflated, delaying the patient from seeing a physician.

One problem with the balloons is that the weight of the balloons can cause them to induce gastric hypertrophy and create gastric erosions, ulcers, lesions, and abrasions within the stomach at the points where the balloon naturally rests due to the large surface area-to-surface area contact. Other problems include infections resulting from bacterial colonization of the gastric balloon and lack of adequate sizing of the balloon prior to deployment in a patient's stomach. Additionally, most gastric balloons have been filled with saline or other liquid, making them heavy and uncomfortable within a patient's stomach. Some gastric balloon designs were introduced in the 1980's, but ultimately were removed from the market.

Publications directed to treatment of obesity include, for example: U.S. Pat. Nos.: 3,406,988 to Moreau et al. for Esophageal Nasogastric Tube; 3,055,371 to Kulick for Device for Regulation and Control of Esophago-Gastric Balloons; 4,133,315 to Berman et al. for Method and Apparatus for Reducing Obesity; 4,246,893 to Berson for Inflatable Gastric Device for Treating Obesity; 4,416,267 to Garren et al. for Method and Apparatus for Treating Obesity; 4,485,805 to Foster Jr. for Weight Loss Device and Method; 4,501,264 to Rockey, for Medical Sleeve; 4,607,618 to Angelchik for Method for Treatment of Morbid Obesity; 4,648,383 to Angelchik for Peroral Apparatus for Morbid Obesity Treatment; 4,694,827 to Weiner et al. for Inflatable Gastric Device for Treating Obesity and Method of Using the Same; 4,739,758 to Lai et al. for Apparatus for Stomach Cavity Reduction; 4,899,747 to Garren et al. for Method and Apparatus for Treating Obesity; 4,908,011 to Jacobsen et al. for Method and Device for Performing a Puncturing Work on an Inflated Balloon-Like Object Implanted in a Patient; 4,930,535 to Rinehold for Folding Leaf Valve and Method of Making; 5,084,061 to Gau et al. for Intragastric Balloon with Improved Valve Locating Means; 5,234,454 to Bangs et al. for Percutaneous Intragastric Balloon Catheter and Method for Controlling Body Weight Therewith; 5,259,399 to Brown for Device and Method of Causing Weigh Loss Using Removable Variable Volume Intragatric Bladder; 5,993,473 to Chan et al. for Expandable Body Device for the Gastric Cavity and Method; 6,454,785 to deHoyas Garza for Percutaneous Intragastric Balloon Catheter for the Treatment of Obesity; 6,579,301 to Bales et al. for Intragastric Balloon Device Adapted to be Repeatedly Wavied in Volume without External Assistance; 6,656,194 to Gannoe et al. for Magnetic Anchoring Device; 6,733,512 to McGhan for Self-Deflating Intragastric Balloon; 6,736,793 to Meyer et al. for Self-Sealing Detachable Balloon; 6,746,640 to Gannoe et al. for Intra-Gastric Fastening Devices; 6,916,307 to Willis et al. for Catheter with Distally Distending Balloon; 6,958,052 to Charlton for Esophageal Balloon Catheter; 7,112,186 to Shah for Gastro-Occlusive Device. Additionally, U.S. Patent Publication Nos. US 2002/0055757 to Torre et al. for Method and Device for Use in Minimally Invasive Placement of Intragastric Devices; US 2003/0158569 to Wazne for Intragastric Device for Treating Morbid Obesity; US 2004/0059289 to Garza Alvarez for Intragastric Balloon Assembly; US 2004/0106899 to McMichael et al for Gastric Balloon Catheter with Improved Balloon Orientation; US 2004/0186502 to Sampson et al. for Self-Inflating Intragastric Volume-Occupying Device; US 2004/0186503 to DeLegge for Intragastric Catheter; US 2004/0267378 to Gazi et al. for Semi-Stationary Balloon in the Gastric Antrim Provided with Connecting an Anchoring Rod for Inducing Weight Reduction in Human Beings; US 2005/0004430 to Lee et al. for Endoscopic Balloon Insertion Device for Treatment of Obesity and Insertion technique of the Same; US 2005/0159769 to Alverdy for Balloon System and Methods for Treating Obesity; US 2006/0129094 to Shah for Gastro-Occlusive Device; US 2006/0155259 to MacLay for Stomach Balloon that can be Inserted and Removed Via Mouth; US 200510167595 to Chen et al. for Methods for Gastric Volume Control. See, also, BioEnterics Intragastric Balloon (BIB™ System), available at www.bioenterics.com and www.bibasia.info.

For these reasons, it would be desirable to provide improved devices and methods for their use in treating overweight and obese patients.

### SUMMARY OF THE INVENTION

An aspect of the invention is directed to a device for treatment of obesity in a patient, including humans and animals. The device comprises an inflatable balloon having a proximal end and a distal end, the inflatable balloon further comprising an interior chamber adapted and configured to receive a filling material; a port in communication with the interior chamber of the inflatable balloon. Furthermore, the inflatable balloon is adapted and configured to assume a predetermined shape in situ and further wherein the inflatable balloon is adapted and configured to have a distal diameter less than or equal to a proximal diameter. Alternatively, in some embodiments, the distal diameter can be greater than or equal to the proximal diameter. The ends of the device can be formed into bulbs and can be connected by a bendable tubular joint.

Yet another aspect of the invention is direct to a device for treatment of obesity in a patient comprising: an inflatable balloon having a proximal end and a distal end, the inflatable balloon further comprising an interior chamber adapted and configured to receive a filling material; a port in communication with the interior chamber of the inflatable balloon wherein the inflatable balloon is adapted and configured to achieve a deployment shape at least partially conformable to an interior dimensional shape of a stomach of the patient and further wherein the inflatable balloon is adapted and configured to have a distal diameter less than or equal to a proximal diameter.

A method for treating obesity according to the invention comprises the steps of introducing a device comprising an inflatable balloon having a proximal end and a distal end, the inflatable balloon further comprising an interior chamber, and a port in communication with the interior chamber of the inflatable balloon wherein the inflatable balloon is adapted and configured to achieve a deployed shape at least partially conformable to an interior dimensional shape of a stomach of the patient; expanding the balloon to provide a conformable geometry; and at least partly filling the chamber of the balloon with a compressible air and/or incompressible fluid. A colored saline can then be used to inflate the balloon in case the balloon ruptures. A rupture can then be detected early from stool, urine or ejected stomach contents.

Yet another method of the invention is directed to a method for deploying a gastric balloon in a patient, the method comprising the steps of: introducing the gastric balloon into a stomach of the patient; and separately inflating a plurality of isolated chambers within the balloon, wherein the chambers have individual volumes such that the collective volume of the chambers remaining inflated after the deflation of any single chamber is such that the balloon is prevented from passing through the pylorus into the small intestine.

Still another method of the invention is directed to a method for selecting a gastric balloon for a patient, the method comprising the steps of: determining an internal volume of a stomach of the patient while the stomach is filled with a biocompatible filling medium; and selecting a balloon having a filling volume less than the determined volume by a preselected amount. Suitable biocompatible filling mediums could include solid, liquid or gaseous materials, or combinations thereof which, in the event of a breach of the system, would not cause harm to the patient.

Another method of the invention is directed to selecting a gastric balloon for a patient. In selecting a gastric balloon for a patient, the healthcare provider determines an internal volume of a stomach of the patient while the stomach is filled with a biocompatible filling medium; and selecting a balloon having a filling volume less than the determined volume by a preselected amount.

Still another method of the invention provides for in vivo monitoring a condition of a gastric balloon. This method comprises the steps of introducing a device comprising an inflatable balloon having a proximal end and a distal end, the inflatable balloon further comprising an interior chamber, a port in communication with the interior chamber of the inflatable balloon wherein the inflatable balloon is adapted and configured to achieve a deployed shape at least partially conformable to an interior dimensional shape of a stomach of the patient, and one or more sensors adapted and configured to sense a condition; and sensing a condition of the device. Additionally, the port can be used to allow pressure within the balloon to be reduced in response to activation by the patient.

Another aspect of the invention is directed to a wireless device for treatment of obesity in a patient. The wireless device comprises: an inflatable balloon having a proximal end and a distal end, the inflatable balloon further comprising an interior chamber adapted and configured to receive a filling material; one or more sensors connected to the inflatable balloon adapted and configured to sense a parameter; and a port in communication with the interior chamber of the inflatable balloon.

Yet another aspect of the invention includes a kit for treatment of obesity in a patient. The kit comprises, for example, a device comprising an inflatable balloon having a proximal end and a distal end, the inflatable balloon further comprising an interior chamber adapted and configured to receive a filling material; one or more sensors connected to the inflatable balloon adapted and configured to sense a parameter; and a port in communication with the interior chamber of the inflatable balloon; an inflation tube adapted and configured to engage the inflatable balloon; one or more delivery materials; and a retrieval device.

An aspect of the invention is directed to an anatomically conformable elongated balloon having an enlarged distal and proximal end. In one embodiment, the distal end is adapted and configured to be smaller than the proximal end. In another embodiment, the distal end is filled with an amount of saline that will achieve comfortable positioning of the device in situ; for example about 100-500 ml saline. The saline filled, distal end is placed in the antrum of stomach. The proximal end is larger, and filled with gas, such as air. The proximal end of the device is positioned within the body of stomach where ingested food is stored, e.g. the upper part of stomach of a patient, where patients include humans and animals. The balloon can be adapted and configured to have one or more chambers. Thus, for example, the chamber of the proximal bulb and the distal bulb are connected and the fluid and gas can flow between the bulbs in response to the position of the patient in order to keep the liquid in the lower part of the stomach. The communication of gas and fluid between sections may reduce any intolerance the patient may have to the gastric balloon placement.

Another aspect of the invention features an elongate balloon adapted and configured to provide two or more prongs or protrusions that extend from the body of the balloon at the distal end. The prongs stabilize and position the balloon above the pylorus. The prongs are further adapted to prevent the balloon from migrating through the pylorus into the duodenum. Gaps or grooves between the prongs allow liquid and other materials to pass through the stomach and avoid obstruction. The proximal end is larger in order to prevent the migration of the balloon into the esophagus. Furthermore, the prongs, bumps or the balloon may be filled with a collapsible material that enables the device to achieve a low profile during delivery, and then assume a deployed configuration that prevents migration through the pylorus. A variety of materials are suitable to achieve this and would be known to persons of skill in the art.

In yet another aspect of the invention, the balloon is made with suitable biocompatible material, such as silicone, that is soft, elastic, and acid resistant. A joint section is provided that makes the overall device performance bendable and able to achieve a predetermined configuration.

In still another aspect of the invention, the surface of at least a portion of the device has bumps, bubbles or dimples, and protrusions extended from the surface. The bumps decrease the actual amount of surface area contact that the device has with the stomach and form grooves or channels there between. In further embodiments, one or two ports will be placed on the proximal end of the balloon for inflating or deflating it.

Another aspect of the invention includes using a biocompatible plastic or metal bar, joint or rod adapted and configured to connect each end of the device, e.g., balloons or bulbs positioned on either end. The bar, joint or rod can be used to support the shape of the device and bend the device at a joint part according to an angle between the gastric antrum and the body. The bar, joint or rod can also be adapted and configured to provide ports which are used to facilitate inflation or deflation of the balloon. In some embodiments, the pH and pressure sensors can be placed on or incorporated into the bar, joint or rod along with a power source, such as a battery. Optionally, the power source can be adapted and configured to be placed in communication with the sensor without necessarily being incorporated into the bar, joint or rod.

One or more sensors can be adapted and configured for use with the various designs of the invention including, for example, a pH sensor inside or outside the balloon that is adapted to detect an early rupture of the balloon; e.g. change of pH within the balloon indicating breach of the balloon and entry of stomach contents. The pH sensor can further be adapted and configured to detect pH in the stomach itself in order to correlate gastric pH and clinical symptoms so that delivery of regimens of antacid can be determined. Further, a pressure sensor can be provided inside the balloon to assist in determining how much to inflate the balloon. The pressure sensor can also be used to detect rupture of the balloon or to provide a guide to how much the patient eats after balloon placement, and whether symptoms of intolerance are caused by gastric spasm or contraction. Each of the sensors can also be adapted and configured to communicate with a device to provide feedback to the user or user's healthcare provider. For example a pager-like device, or a watch (similar to a heart rate monitor watch) can be configured to receive a signal from one or more sensors. The information from the sensor can then result in an alarm sounding, or a read-out display change. Additionally, in some instances, the sensor can communicate to the external device which in turn communicates with a health care provider when appropriate. Additionally, a release button, lever, switch, or toggle, can be used to allow the patient to release pressure in the device when needed. The release button is typically in communication with a sensor in proximity to the access valve. Once activated the release button sends a signal to the sensor in proximity to a two-way valve or an outflow valve. The sensor causes the valve to open thereby relieving pressure. The release button can be manually actuable and/or can be in wireless communication with the sensor.

To deploy the device, the balloon can be placed within the stomach during an outpatient upper endoscopy procedure with conscious sedation by a gastroenterologist. The procedure takes 15 to 30 minutes. First, a routine upper endoscopy is performed and an overtube is introduced through the endoscopy into the esophagus. The endoscopy is pulled out. A device containing the balloon is inserted into the stomach via the overtube and the balloon is deployed in the stomach.

The size of the balloon can be altered in situ if the goal of weight loss is not achieved. Altering the size of the device in situ can be achieved by, for example, activating a size controlling chamber. The balloon also can be removed if the ideal weight loss is met or complications develop.

In some embodiments, the size of the proximal balloon and the distal balloon can be altered according to the size of the patient's stomach. In still other embodiments, a third balloon may be attached to the proximal balloon to achieve a space occupying effect as well.

The balloon can be inflated with a mixture of gas and fluid in either one chamber, more than one chamber, or each of a plurality of chambers as appropriate based on the design of the device. The ratio of fluid and gas in any of the chambers can be altered according to the patient's symptoms and efficacy of weight loss.

Another aspect of the invention is directed to an intragastric balloon device comprising a hollow pliable sac defining an interior space, the sac further comprising a proximal end and a distal end and an exterior surface; at least one protrusion extending from the exterior surface of the sac; and an access port adapted to provide material access to the interior space. Additionally, the device may further comprise at least one prong extending from the exterior surface of the sac at the distal end. The interior space of the device may be divided into two isolated chamber adapted and configured to be filled with a material. In such a case, the device may further comprise an access port wherein the access port is configured to provide material access to the at least one of the isolated chambers. The device may further comprise a second access port. In such an embodiment, a first access port can provide material access to a first chamber in the interior space and the second access port provides material access to a second chamber in the interior space. The device may further comprise at least one prong extending from the exterior surface of the sac at the proximal end. In some embodiments, the device may further comprise one or more sensors that are adapted and configured to be positioned on the exterior surface of the sac. The sensor is typically adapted and configured to communicate wirelessly with a data receiver. In some aspects, the device the distal end of the device has a smaller diameter than the proximal end of the device. The diameter of the sac varies with the length of the sac. Typically, the diameter of the sac at the proximal end is greater than the diameter of the sac at the distal end.

In yet another aspect of the device, the device is an intragastric balloon device for positioning in the stomach comprising: a hollow pliable sac defining an interior space, the sac further comprising a proximal end and a distal end and an exterior surface; a series of protrusions extending from the exterior surface of the sac; an access port adapted to provide material access to the interior space; and a series of prongs located on the distal end of the sac, wherein the prongs are adapted to prevent migration of the sac. The interior space may further comprise at least two isolated chambers adapted and configured to be filled with a material. Additionally, the device may further include an access port wherein the access port is configured to provide material access to the at least one isolated chamber. Alternatively, the device may comprise a second access port. The first access port provides material access to a first chamber in the interior space and the second access port provides material access to a second chamber in the interior space. Typically, the distal end of the device has a smaller diameter than the proximal end of the device. In some embodiments, at least one prong may extend from the exterior surface of the sac at the proximal end. In a further aspect of the device, a sensor may be included with the device. Typically, the sensor is adapted and configured to be positioned on the exterior surface of the sac. The sensor can be further adapted and configured to communicate wirelessly with a data receiver. The diameter of the device varies with the length of the sac. Typically the diameter of the sac at the proximal end is greater than the diameter of the sac at the distal end.

An intragastric balloon device comprising: a hollow pliable sac, the sac further comprising a proximal end and a distal end and an exterior surface; an interior space defined within the sac wherein the interior space further comprises at least two isolated chambers wherein the isolated chambers are adapted to be filled with a media; and a controlling mechanism in communication with the exterior surface of the sac wherein the controlling mechanism is adapted to conform and maintain the device in a user defined configuration. In some aspects, the invention further comprises at least one prong extending from the exterior surface of the sac at the distal end. The device may additionally comprise an access port wherein the access port is adapted and configured to provide material access to the at least one isolated chamber. In some embodiments, a second access port is included with the device. The first access port provides material access to a first chamber in the interior space and the second access port provides material access to a second chamber in the interior space. The distal end of the device has a smaller diameter than the proximal end of the device. The distal end may further comprise at least one prong extending from the exterior surface of the sac at the proximal end. In a further aspect of the device, a sensor is included with the device. The sensor is typically adapted and configured to be positioned on the exterior surface of the sac. Additionally, the sensor can be adapted and configured to communicate wirelessly with a data receiver. In some embodiments, the diameter of the sac varies along the length of the sac. In such an embodiment, the proximal diameter of the sac at the proximal end is greater than the diameter of the sac at the distal end.

An aspect of the device is directed to an intragastric balloon device comprising: a first inflatable chamber; a second inflatable chamber; and an elongate neck region connecting the first and second inflatable chambers wherein the neck region is adapted and configured to conform and maintain the device in a user defined configuration. The device may further comprise at least one prong extending from the exterior surface of the sac at the distal end. Additionally, the device may include an access port wherein the access port is configured to provide material access to the at least one chamber. In some aspects, a second access port is included with the device. The first access port provides material access to a first chamber in the interior space and the second access port provides material access to a second chamber in the interior space. Typically, the first inflatable chamber of the device has a smaller diameter than the second inflatable chamber of the device. In addition, at least one prong may extend from the second inflatable chamber wherein the second inflatable chamber is located proximal to the pylorus of a patient. In some embodiments, the device further comprises a sensor. The sensor is typically adapted and configured to be positioned on the exterior surface of the sac. Additionally, the sensor can be adapted and configured to communicate wirelessly with a data receiver.

Where desired, a diet and psychological counseling should be carried out before considering the gastric balloon placement. If patient can achieve some degree of weight loss in about 3 months, a gastric balloon can be placed. Typically, continued diet control and behavior monitoring during, and after the gastric balloon placement are performed to maintain long-term benefits.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a cross-sectional side view of the stomach of a human.

FIG. 2 illustrates an embodiment of an intragastric balloon device having a single interior chamber and a shape that is at least partially conforming to the interior shape of the stomach comprising dimples and a pyloric prong.

FIGS. 3A-D illustrate an embodiment of an intragastric balloon device having two interior chambers and a shape that is at least partially conforming to the interior shape of the stomach comprising dimples and a pyloric prong;

FIGS. 4A-D illustrate another embodiment of an intragastric balloon device featuring a shape control mechanism;

FIG. 5 illustrates an embodiment of an intragastric balloon device comprising a proximal inflatable chamber, a distal inflatable chamber and a neck portion connecting the two chambers;

FIG. 6 illustrates an embodiment of an intragastric balloon device comprising a plurality of proximal inflation chambers;

FIG. 7 illustrates an embodiment of an intragastric balloon device having proximal prongs on the proximal end and distal prongs on the distal end;

FIG. 8 illustrates an embodiment of an intragastric balloon device having a proximal balloon and a distal balloon with a flexible neck region there between;

FIG. 9 illustrates a patient with a device implanted where the device is in wireless communication with a personal display and optionally in remote communication with a physician's office;

FIG. 10 illustrates a flow chart of a method for deploying the device; and

FIG. 11 illustrates a flow chart of a method for treating a patient with the device.

### DETAILED DESCRIPTION OF THE INVENTION

A non-surgical, easy to perform, reversible, low cost procedure is contemplated under the invention as a way to treat moderately obese patients, patients who are contraindicated for surgery, or for use as a bridge procedure before bariatric surgery. With the rise of childhood obesity, gastric bypass surgeries are currently being performed in children. However, the long term benefits and complications, including malabsorption, are still unknown. As discussed above, placing a balloon in the stomach to induce satiety has been used in countries outside the United States. During these procedures, a round balloon with 500 ml of saline is placed in the stomach through an upper endoscopy. The procedure has been safe and has achieved weight loss for some moderate obesity patients. However, good clinical data is lacking and the balloon has failed to achieve a significant weight loss. Additionally, the devices and procedures may be suitable for use in animals.

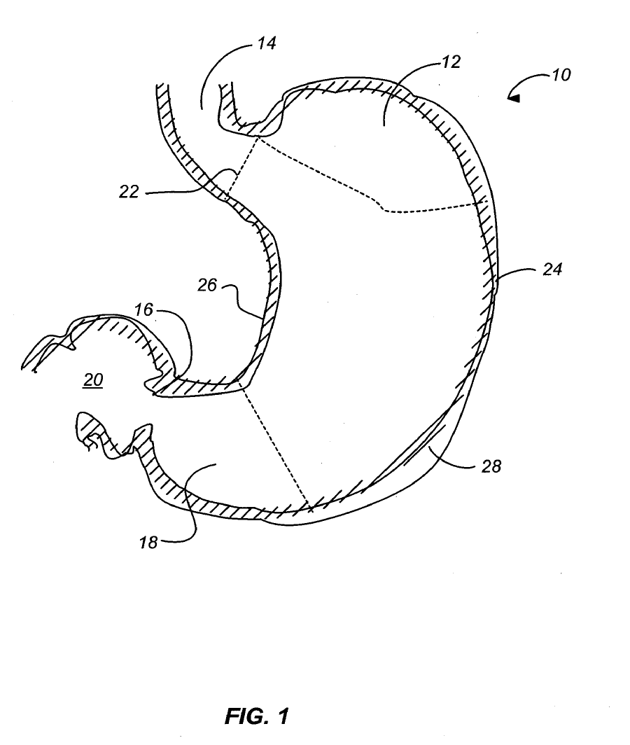

In order to understand the context of the device, it is helpful to appreciate an anatomical framework for the device. Accordingly, FIG. 1 illustrates a cross-sectional side view of the stomach of a human. The stomach 10 is accessed via the esophagus 14. The stomach 10 is continuous with the esophagus 14 at the cardia 22 and with the duodenum 20 (part of the small intestinal tract) at the pylorus 16. The lesser curvature 26 lies on the posterior surface of the stomach and is the downward continuation of the posterior wall of the esophagus. Proximal the pylorus 16 the area of the stomach wall corresponding to the lesser curvature 26 curves upwards to complete a “J” shape. Where the esophagus joins the stomach, the interior region angles acutely upwards and curves downward to form the greater curvature 28. The wall then curves upwards slightly towards the pylorus 16.

The stomach 10 itself is divided into three regions: the fundus 12, the body 24, and the antrum 18. Typically, stomach size varies with the volume of the food it contains. The size of the stomach can be 1.5 L or more in an adult. Typically the stomach can hold from 2 L to 4 L of food. When a meal is eaten, food accumulates in the stomach, with the last portion of the meal remaining in the fundus 12. Food is mixed with gastric juices while in the stomach and the gastric muscles contract which causes a churning movement. The stomach normally functions to temporarily store food to allow digestive enzymes to act. During this time, chemical digestion and mechanical break down occurs.

### I. Devices

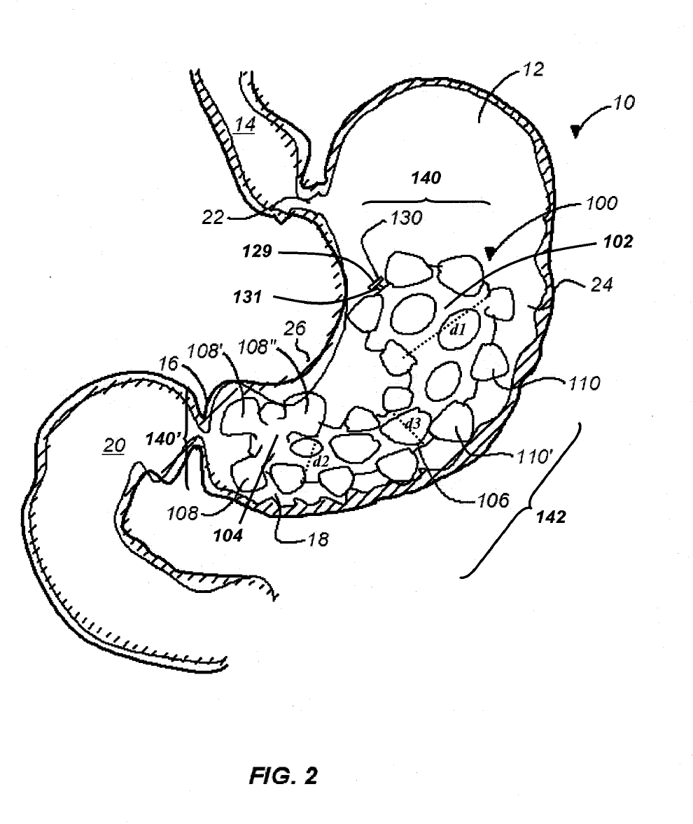

Turning now to the embodiment of the invention, FIG. 2 illustrates an embodiment of an intragastric device 100 suitably adapted and configured to be positioned within a stomach of a patient. The device is adapted and configured for treatment of obesity, weight loss, etc. The device 100 has a proximal end 102 and a distal end 104. The proximal end 102 is positioned nearest the cardia 22 while the distal end 104 is positioned near the pylorus 16. The device 100 has a single interior chamber and one or more discrete bumps, bubbles or dimples 110, 110′ on the exterior surface 106. The exterior bumps 110, 110′ are configured to extend away from the surface of the device 100 to reduce the total surface area of the device that contacts the interior wall of the stomach. Thus, a device having a total surface area of, for example, 800 cm², can be configured so that less than 50% of the surface area of the device can contact the walls of the stomach, preferably, less than 40%, and more preferably less than 30%. Additionally, the dimples allow food to pass along the length of the device, e.g. through grooves or valleys formed between the dimples, on all sides from the fundus to the antrum without being impeded by the device (e.g., where the device wall would otherwise completely engage the wall of the stomach). The prongs, bumps, or the balloon itself, may be formed from or filled with a collapsible material that enables the device to achieve a low profile during delivery, and then assume a deployed configuration that prevents migration through the pylorus. A variety of materials are suitable to achieve this function and would be known to persons of skill in the art. Furthermore, shape memory materials can be used to make the device such that after deployment the device assumes a shape that prevents it from traveling into the intestines in the event of a damage to the device.

A one way valve 130 can be incorporated that enables air and fluid to be placed within the chamber of the balloon. The one way valve can be used with a glue-like material that seals the port after access. In some embodiments, the one way valve can be configured such that it has a neck 129 and a cap 131 that facilitate capturing the device with a removal tool to remove the device after the treatment protocol is completed. Alternatively, a two way valve can be used. Additionally, the ability to capture the valve will also facilitate further inflating or deflating the device, during a treatment protocol, if necessary. In some embodiments, the port can function in communication with a release button, such that when a patient begins to feel uncomfortable, e.g., due to an increase in pressure within the balloon, an actuatable button is pressed which reduces the pressure within the balloon by releasing balloon contents. When a patient begins to feel discomfort due to a change in pressure in the device as caused by, e.g., change in pressure due to altitude, a release button can be pressed to relieve pressure. The release button is typically located on the monitoring device and can be pressed by the patient when desired. The release button is in communication with a receiver located in proximity to a release valve located on the surface of the device. Alternatively the receiver in communication with the device is located near the access valve. In such an embodiment, the access valve is typically a two-way valve. Furthermore, the receiver can be wirelessly connected to the device.

Additionally, as shown in the embodiment of FIG. 2 a series of distal projections 108, 108′, 108″ are provided that extend away from the exterior surface 106 of the device. The distal projections 108 serve to engage the stomach near the pylorus 16 such that the projections 108 prevent the device 100 from entering the small intestine 20, and simultaneously preventing the body of the device from engaging the pyloric channel to block off the pylorus or otherwise prevent food from passing from the stomach into the intestinal tract.

The device 100 is adapted and configured such that it is has a tendency to adopt an overall profile that conforms to at least a portion of a profile of an interior dimension of a stomach, as is apparent from the view of the device in situ shown in FIG. 2. The device 100 can be adapted and configured such that the device has two bulbous ends 140, 140′, one proximal one distal, such that the diameter d1 of the proximal bulbous end 140 is greater than the diameter d2 of the distal bulbous end 140′. Further, the diameter d3 of a neck region 142 positioned between two bulbous ends 140, 140′ can be smaller than the diameter of the other two regions at a midpoint along its length, or no larger than the diameter of the distal end. The length of the neck section can vary from 4 cm to greater than 10 cm.

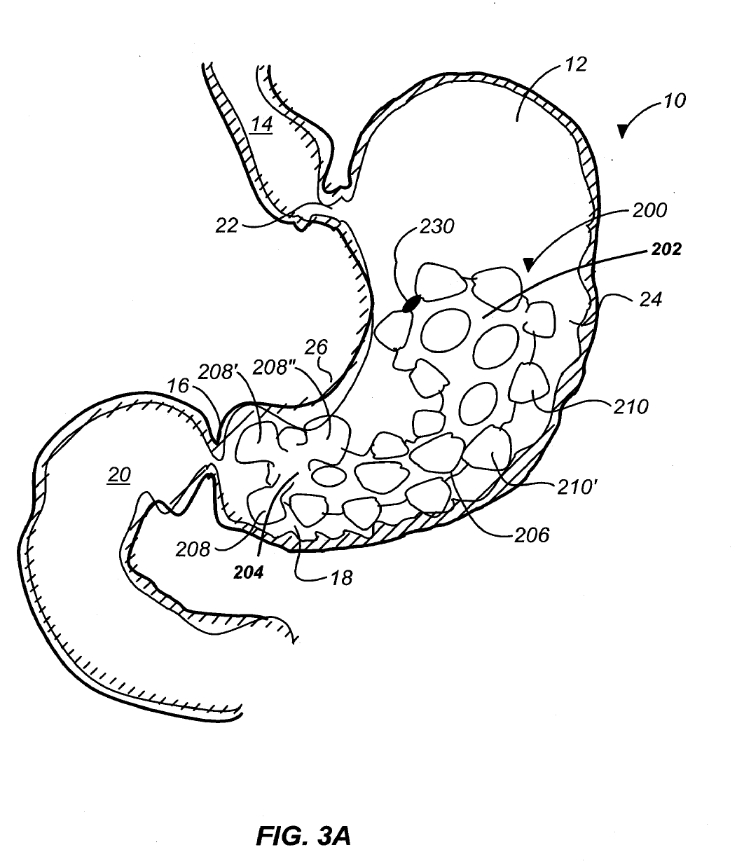

Turning now to the embodiments of the invention, FIGS. 3A-D illustrate an embodiment of an intragastric device 200 suitably adapted and configured to be positioned within a stomach of a patient. The device is adapted and suitable for treatment of obesity, weight loss, etc. The device 200 has a proximal end 202 and a distal end 204. The proximal end 202 is positioned nearest the cardia 22 while the distal end 204 is positioned near the pylorus 16. The device 200 has one or more inflatable sections or chambers and one or more discrete bumps, bubbles or dimples 210, 210′ on the exterior surface 206. The exterior bumps 210 are configured to extend away from the surface of the device 200 to reduce the total surface area of the device that contacts the interior wall of the stomach. A device similarly sized to the device of FIG. 3 is contemplated.

Additionally, as shown in the embodiment of FIG. 3A a series of distal projections 208, 208′, 208″ are provided that extend away from the exterior surface 206 of the device. The distal projections 208 serve to engage the stomach near the pylorus 16 such that the projections 208 prevent the device 200 from entering the small intestine 20, while preventing the body of the device from engaging the pyloric channel to block off the pylorus or otherwise prevent food from passing from the stomach into the intestinal tract.

Turning now to FIG. 3B, a cross-section of the device 200 is depicted. As illustrated in the cross-sectional view, the device is internally separated into two chambers 220, 222. The interior chambers 220, 222 are accessed via an access port or inflation port 230 having a lumen that communicates with each of the chambers. As will be appreciated by those skilled in the art, the access port can be configured such that a first and second port is provided at a single location (as depicted). Alternatively, the device can be configured such that each chamber has a separate access port in direct communication with an exterior surface of the device. Turning back to the access port configuration depicted, an inner access port 232 with an elongated tubular section is provided that extends from an exterior surface 206 of the device 200 through the first chamber 220 and through an intermediate wall or divider 224 separating the two chambers and then into the second chamber 220. The inner access port 232 enables the second chamber 222 to be filled partially or completely with suitable material. As shown in FIG. 3D, a second access port 234 is positioned around the inner access port 232 and has a tubular access lumen that enables fluid or material to be filled within the first chamber 220. Each of the access ports is adapted and configured to provide a valve mechanism to allow fluid or material to be inserted into the chamber with which the access port communicates. Typically, the valve is a two way valve, allowing fluid or material to be placed within the chamber and removed from the chamber. As discussed above, a panic button feature can be provided that allows a patient to reduce the pressure within the device by allowing the filling material to pass out of the device. As will be appreciated by those skilled in the art, additional chambers can be provided without departing from the scope of the invention. Sensors 260, 260′ are provided to sense a change in condition in each of the chambers; or to sense a condition exterior the device, such as a patient condition or pH of the stomach contents. Sensors can be placed on the ends 220, 222 or in the middle 242.

The device 200 is adapted and configured such that it is has a tendency to adopt an overall profile that conforms to at least a portion of a profile of an interior dimension of a stomach, as is apparent from the view of the device in situ shown in FIG. 3A. The device 200 can be adapted and configured such that the device has two bulbous ends 240, 240′, one proximal one distal, such that the diameter d1 of the proximal end 240 is greater than the diameter d2 of the distal end 240′. Further, the diameter d3 of a neck region 242 positioned between two bulbous ends 240, 240′ can be smaller than the diameter of the other two regions at a midpoint along its length, or no larger than the diameter of the distal end. The length of the neck section can vary from 4 cm to greater than 10 cm. FIG. 3c is a view of the device from a distal end illustrating an example of a relative diameter relationship in an embodiment between the distal end, the neck region and the proximal end. FIG. 3D is a view of the device from the proximal end. As the diameter of the neck region and the distal end is smaller than the proximal end, from the proximal view, the other sections of the device would not be seen in this view. A potential location for the access port is shown on a proximal surface of the device, which would be near the cardia. Additionally, separate access ports for each interior chamber are illustrated. Positioning the access port in this location facilitates accessing the device to either add additional material or remove material and deflate the device, e.g. for removal from the stomach through the esophagus.

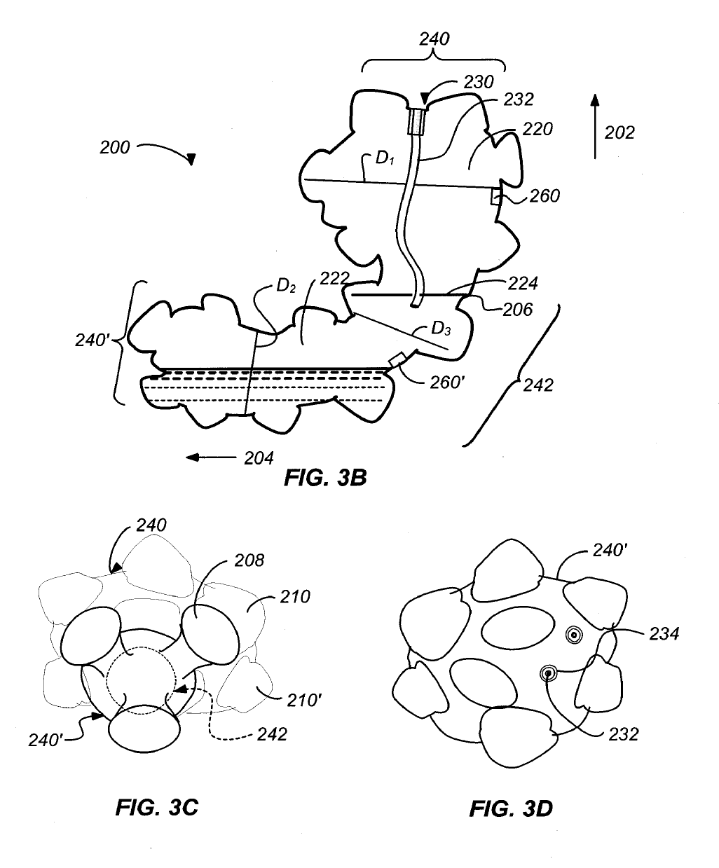

FIGS. 4A-D illustrates yet another embodiment of an intragastric device 300 suitable for treatment of obesity and weight loss. The device 300 depicted in FIG. 4 features many of the same structural components as the devices of FIGS. 2-3.

The device 300 has a proximal end 302 and a distal end 304. The proximal end 302 is positioned nearest the cardia 22 while the distal end 304 is positioned near the pylorus 16. The device 300 has one or more inflatable sections or chambers and one or more discrete bumps, bubbles, dimples, or protrusions 310, 310′ on the exterior surface 306. In between the bumps, grooves 311 or valleys are formed. The exterior bumps 310 are configured to extend away from the surface 306 of the device 300 to reduce the total surface area of the device that contacts the interior wall of the stomach. Additionally, as shown in the embodiment of FIG. 4A a series of distal projections 308, 308′, 308″ are provided that extend away from the exterior surface 306 of the device. The distal projections 308 serve to engage the stomach near the pylorus 16 such that the projections 308 prevent the device 300 from entering the intestinal tract 20, while preventing the body of the device from engaging the pylorus to block off the pyloric channel or otherwise prevent food from passing from the stomach into the intestinal tract.

Turning now to FIG. 4B a cross-section of the device 300 is depicted. As illustrated in the cross-sectional view, the device is internally separated into two chambers 320, 322. The interior chambers 320, 322 are accessed via an access port or inflation port 330 having a lumen that communicates with each of the chambers. As will be appreciated by those skilled in the art, the access port can be configured such that a first and second port is provided at a single location (as depicted) or can be configured such that each chamber has a separate access port in direct communication with an interior surface of the device. Turning back to the access port configuration depicted, an inner access port 332 with an elongated tubular section is provided that extends from an exterior surface 306 of the device 300 through the first chamber 320 and through an intermediate wall or divider 324 separating the two chambers and then into the second chamber 322. The inner access port 332 enables the second chamber 322 to be filled partially or completely with suitable material. A second access port 334 (shown in FIG. 4D) is positioned around the inner access port 332 and has a tubular access lumen that enables fluid or material to be filled within the first chamber 320. Each of the access ports is adapted and configured to provide a valve mechanism to allow fluid or material to be inserted into the chamber with which the access port communicates. Typically, the valve is a two way valve, allowing fluid or material to be placed within the chamber and removed from the chamber. As discussed above, a panic button feature can be provided that allows a patient to reduce the pressure within the device. As will be appreciated by those skilled in the art, additional chambers can be provided without departing from the scope of the invention. Two separate access ports 332, 334 are depicted.

The device 300 additionally features a shape controlling mechanism 350. The shape controlling mechanism 350 facilitates deploying the device 300 into a shape that is maintained in situ. Additionally, the shape controlling mechanism helps to prevent rotation of the device within the stomach which, when combined with other features, can resist the tendency of the device to migrate proximally toward the pylorus, for example, when a patient is prone. The mechanism 350 has an elongated tube 352 that extends at least a portion of the length of the device 300. The elongate tube 352 can be hollow or solid, flexible, or made of shape memory material that achieves its shape after deployment, e.g. as a result of temperature. Additionally, the proximal and distal ends 354, 354′ of the shape controlling mechanism 350 can be further adapted to provide diameter control of one or both of the ends of the device. In another embodiment, the shape controlling mechanism 350 can be adapted and configured such that it is integral to the body of the device to form a spine. The integrally formed parts can be configured such that they act in a unified manner, or such that separate components are formed together into a single device. In yet another embodiment, the shape controlling mechanism can be adapted to also function partially as the access port. Radiopaque markers 360 can be provided in order to facilitate assessment of the positioning of the device in situ. The markers 360 can, for example, be incorporated into or associated with the wall of the device.

FIG. 4c is a view of the device from a distal end illustrating an example of a relative diameter relationship in an embodiment between the distal end, the neck region and the proximal end. FIG. 4D is a view of the device from the proximal end. As the diameter of the neck region and the distal end is smaller than the proximal end, from the proximal view, the other sections of the device would not be seen in this view. A potential location for the access port is shown on a proximal surface of the device, which would be near the cardia. Additionally, separate access ports for each interior chamber are illustrated. Positioning the access port in this location facilitates accessing the device to either add additional material or remove material and deflate the device, e.g. for removal from the stomach through the esophagus.

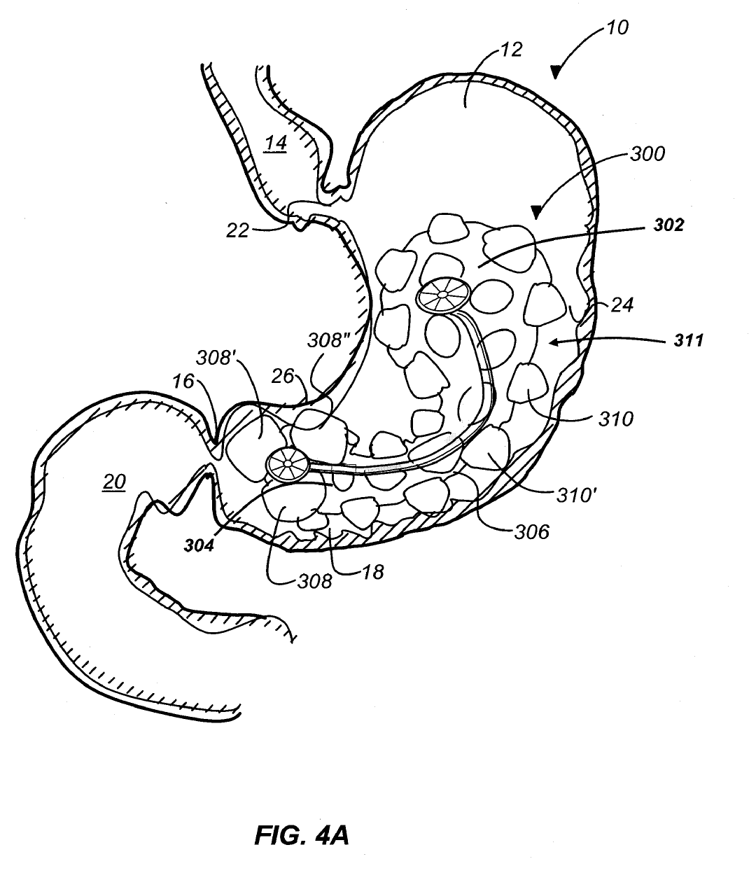

FIG. 5 illustrates yet another embodiment of an intragastric device suitable for treatment of obesity and weight loss. The device 400 depicted in FIG. 5 features many of the same structural components as the devices of FIG. 2-4.

As with the previous embodiments, the device 400 has a proximal end 402 and a distal end 404. The proximal end 402 is positioned nearest the cardia 22 while the distal end 404 is positioned near the pylorus 16. The device 400 has one or more inflatable sections or chambers and one or more discrete bumps, bubbles or dimples 410, 410′ on the exterior surface 406. The exterior bumps 410 are configured to extend away from the surface of the device 400 to reduce the total surface area of the device that contacts the interior wall of the stomach. Additionally, as shown in the embodiment of FIG. 5, a series of distal projections 408, 408′, 408″ are provided that extend away from the exterior surface 406 of the device. The distal projections 408 serve to engage the stomach near the pylorus 16 such that the projections 408 prevent the device 400 from entering the intestinal tract 20, while preventing the body of the device from engaging the sphincter to block off the sphincter or otherwise prevent food from passing from the stomach into the intestinal tract.

In this embodiment, the device 400 is formed from a plurality of separate components each having its own interior chamber, e.g. balloons. A first chamber 420 and a second chamber 422 are provided. Each chamber is connected by a neck region 442. The distal end has radial projections 408. As will be appreciated by those skilled in the art, the one or more the pieces can be formed integrally with one or more other pieces, or can be formed from separate pieces in communication with an adjacent piece.

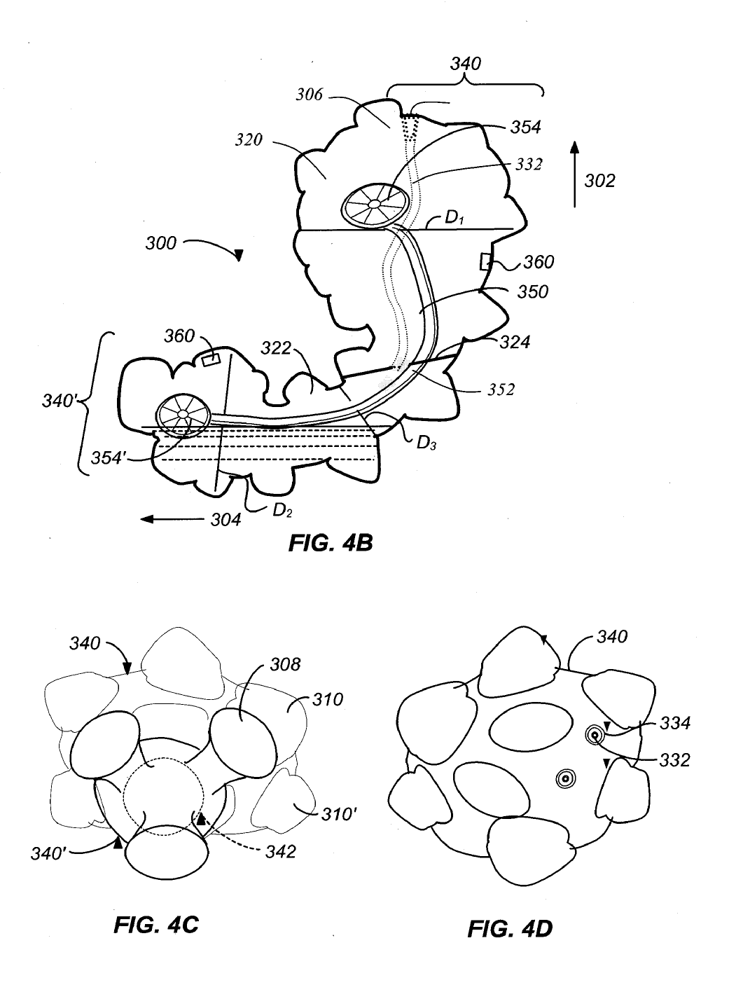

FIG. 6 illustrates still another embodiment of an intragastric device 500 suitable for treatment of obesity and weight loss.

Again, the device 500 has a proximal end 502 and a distal end 504. The proximal end 502 is positioned nearest the cardia 22 while the distal end 504 is positioned near the pylorus 16. The device 500 has one or more inflatable sections or chambers and one or more discrete bumps, bubbles or dimples 510, 510′ on the exterior surface 506. The exterior bumps 510 are configured to extend away from the surface of the device 500 to reduce the total surface area of the device that contacts the interior wall of the stomach and to create grooves 511 or valleys in between the bumps. Additionally, as shown in the embodiment of FIG. 6 a series of distal projections 508, 508′, 508″ are provided that extend away from the exterior surface 506 of the device. The distal projections 508 serve to engage the stomach near the pylorus 16 such that the projections 508 prevent the device 500 from entering the intestinal tract 20, while preventing the body of the device from engaging the sphincter to block off the sphincter or otherwise prevent food from passing from the stomach into the intestinal tract.

As is further appreciate, the device 500 disclosed in FIG. 6 has a proximal end 540 comprised of a plurality of inflatable sections 540, 540′, 540″. This embodiment is illustrated with the inflatable sections being contained within each other in a first chamber 520. Each balloon can be separately engaged so that the inner balloon is inflated separately from an intermediate balloon and an exterior balloon. However, other configurations can be employed without departing from the scope of the invention. In the embodiment depicted in this figure, the plurality of proximal balloons can be separately engaged to provide incremental control over the size of the proximal end of the balloon. For example, a single balloon can be inflated. If the single balloon inflation does not achieve the desired results, then the device can be engaged and a second balloon can be inflated. This process can be repeated as often as desired to achieve the desired result. In some embodiments a plurality of inflatable sections may be located within the second chamber 522.

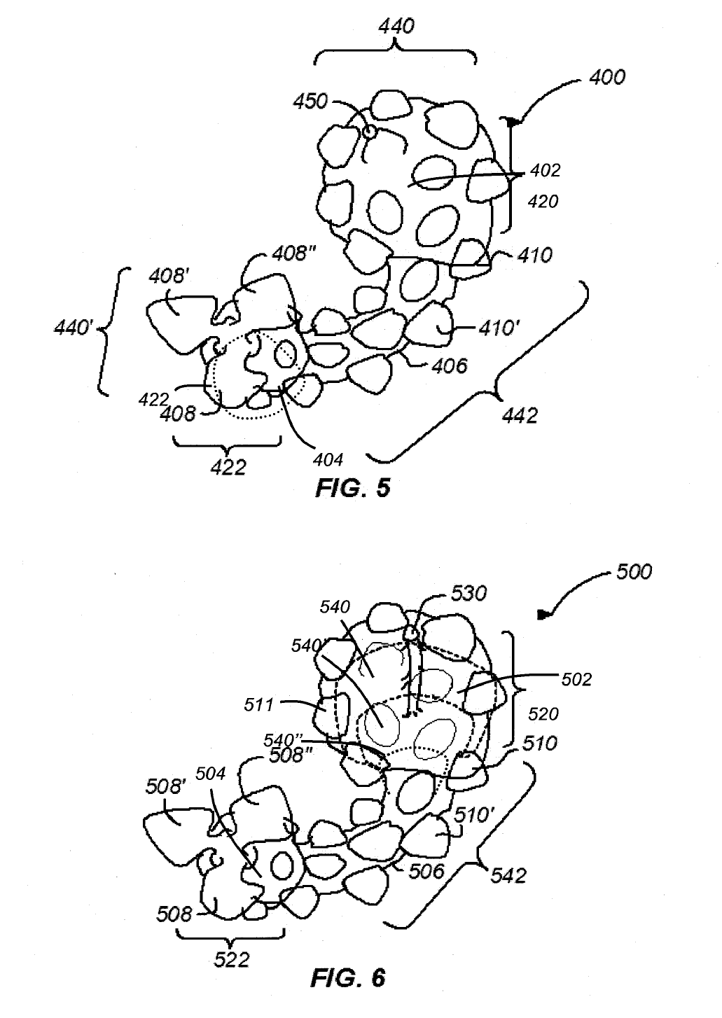

FIG. 7 illustrates yet another embodiment of an intragastric device 600 suitable for treatment of obesity and weight loss. Again, the device 600 has a proximal end 602 and a distal end 604. The proximal end 602 is positioned nearest the cardia 22 while the distal end 604 is positioned near the pylorus 16. The device 600 has one or more inflatable sections or chambers and one or more discrete bumps, bubbles or dimples 610, 610′ on the exterior surface 606. The exterior bumps 610 are configured to extend away from the surface of the device 600 to reduce the total surface area of the device that contacts the interior wall of the stomach.

Additionally, as shown in the embodiment of FIG. 7 a series of distal projections 608, 608′, 608″ are provided that extend away from the exterior surface 606 of the device. The distal projections 608 serve to engage the stomach near the pylorus 16 such that the projections 608 prevent the device 600 from entering the intestinal tract 20, while preventing the body of the device from engaging the pylorus to block off the pyloric channel or otherwise prevent food from passing from the stomach into the intestinal tract.

In the embodiment depicted in FIG. 7, the proximal end is also configured to provide two or more projections 670, 670′, 670″ in proximity to a sensor 650 adapted and configured to engage the side walls of the stomach in the antrum region and the fundus region. Providing fundus or proximal projections further assists in preventing the device from migrating toward the cardia when the patient changes position, e.g. to a prone position.

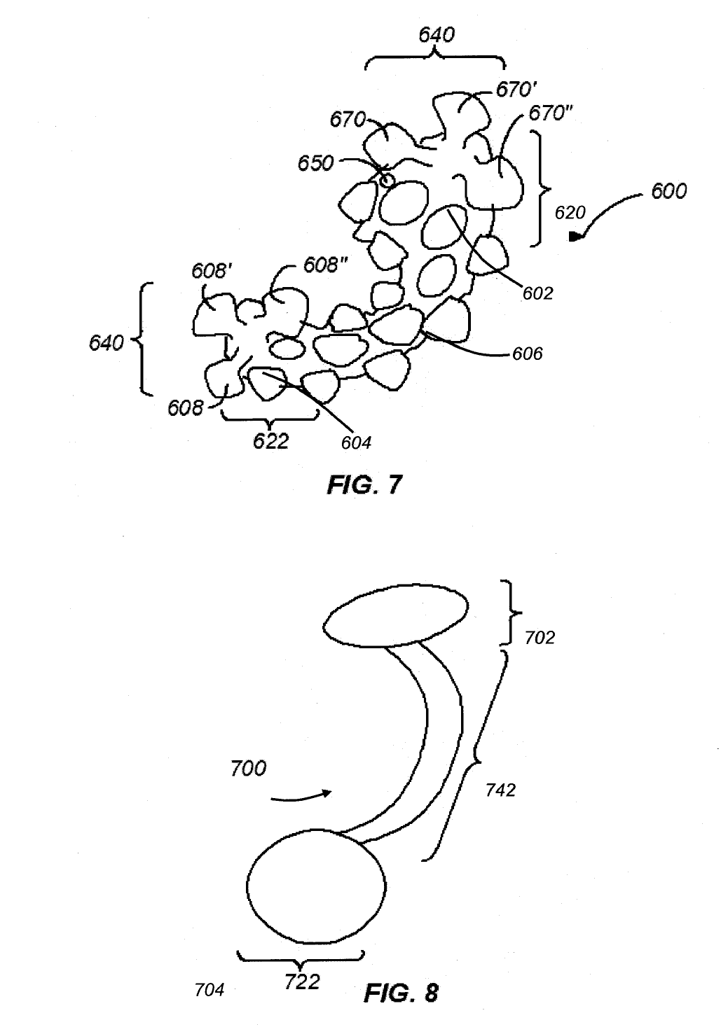

FIG. 8 illustrates yet another embodiment of an intragastric device 700 suitable for treatment of obesity and weight loss. Again, the device 700 has a proximal end 702 and a distal end 704. The proximal end 702 is positioned nearest the cardia 22 while the distal end 704 is positioned near the pylorus 16. The device 700 can be configured to have a single inflatable chamber or a plurality of inflatable sections or chambers. This embodiment is depicted with an elongate, thin neck region 742. The thin neck region facilitates placement of the device while reducing the amount of area of the device that contacts the stomach wall, while providing a bendable, conformable section that facilitates adapting the shape of the device to the interior dimensions of a target patient's stomach chamber. This embodiment may be further modified by providing any combination of one or more bumps or prongs as described with previous embodiments.

The various embodiments of the devices described above can also be provided with radiopaque markers on the wall of the balloons to facilitate assessment of the positioning of the device in situ using a variety of imaging techniques including but not limited to ultrasound, fluoroscopy and/or x-rays.

### II. Materials

Materials suitable for making the devices, or components of the devices, described herein would be apparent to those of skill in the art and include, but is not limited to biocompatible metals (such as cobalt chromium steel, surgical steels, titanium, titanium alloys, tantalum, tantalum alloys, aluminum, etc.), ceramics, polyethylene, biocompatible polymers, and other materials known in the orthopedic arts. Furthermore, surfaces may be formed from biocompatible metals such as cobalt chromium steel, surgical steel, titanium, titanium alloys (such as the nickel titanium alloy Nitinol), tantalum, tantalum alloys, aluminum, etc. Shape memory alloys, such as Nitinol, can also be used to facilitate deployment and flexibility of the device.

In some embodiments, portions of the device can also be formed from suitable polymers include polyesters, aromatic esters such as polyalkylene terephthalates, polyamides, polyalkenes, poly(vinyl) fluoride, PTFE, polyarylethyl ketone, and other materials that would be known to those of skill in the art. Various alternative embodiments of the devices and/or components could comprise a flexible polymer section (such as a biocompatible polymer) that is rigidly or semi rigidly fixed.

In each of the embodiments depicted, the device should be constructed from durable, biocompatible material. The proximal chamber of the device will be filled with material of lower density than the material in the distal chamber to facilitate placement of the device within the stomach. For example, the proximal chamber can be filled with air while the distal chamber is filled with fluid. Additionally, a third chamber can be provided in a middle region of the device which is configured to position the device while a patient is in a prone position such that the device will not migrate up toward the esophagus. The third chamber can extend radially away from the device and be weighted at its distal end (e.g., the end furthest away from the central body of the device).

Additionally, polymers can be employed that use polymer surface modification. For example, Oligomeric Surface-Modifying End Groups (SME) can be appended to the polymers used for the device. As would be appreciated by those skilled in the art, the SME are appended to base polymers during synthesis. The end groups are surface active and therefore migrate to the surfaces of formed articles, e.g. membranes, coatings and catheters. A wide range of inert (e.g., hydrocarbon, fluorocarbon, silicone, PEO, etc.) or bioactive groups can also be incorporated into polymers with little or no change in bulk physical properties or processability. Another feature of the SME-containing polymers is that it adds mobility of end groups relative to backbone groups which may facilitate self assembly of molecular overlayers by the surface-modifying groups.

In some components it may be useful to employ, for example, a family of dense (i.e. without permanent pore structure) selectively-permeable membranes which can be made permeable to high-molecular-weight solutes, such as proteins. With these materials, the permeability coefficient and molecular weight cutoff can be varied through changes in the composition and the molecular structure of the membrane polymer during synthesis. Suitable elastomeric, optically-transparent membranes and coatings may be used for the devices or components of the device that are cast from organic solvents or water-based dispersions, or they may be extruded from the melt. In vivo testing of a hybrid artificial pancreas by PTG resulted in virtually no fibrous tissue encapsulation around these very hydrophilic membranes. Rather the implants became surrounded by well-vascularized tissue with new capillaries forming within one or two cell layers of the membrane surface. The membranes have applications in cell culture as gas and nutrient-permeable substrates and/or microbial barriers, in immunoisolation and in (protein-based) controlled released.

Suitable materials would be appreciated by those skilled in the art, and when reviewing, for example, U.S. Patent Publ. 2005/0282997 to Ward et al. for Control of Polymer Surface Molecular Architecture Via Amphiphathic Endgroups; 2004/0140264 to Ward et al. for Production of Potable Liquids. See also, U.S. Pat. Nos. 7,157,528 to Ward for Permselective Structurally Robust Membrane Material; 6,692,528 to Ward et al. for Devices that Change Size/Shape Via Osmotic Pressure; 5,756,632 to Ward et al. for Systems for Permeating Molecules of Predetermined Molecular Weight Range; 5,589,563 to Ward et al. for Surface-Modifying Endgroups for Biomedical Polymers; 5,482,123 to Ward et al. for Copolymers and Non-porous, Semi-permeable Membrane Thereof and its Use for Permeating Molecules of Predetermined Molecular Weight Range; 5,190,546 to Jervis for Medical Devices Incorporating SIM Memory Alloy Elements; and 5,964,770 to Flomenblit for High Strength Medical Devices of Shape Memory Alloy.

### III. Device Operation

After a device of the invention, or its components, has been inflated and left in place within a patient, it may become desirable to adjust the size and/or buoyancy. Such adjustment may be desirable for patient comfort, efficacy, or other reasons. To perform such adjustments, the device is accessed, for example using an endoscope adapted to provide a suitable working tool. For example, the device may be grasped with graspers and an inflation tubes may be suitably attached or docked to the inflation port or ports accessible from the exterior of the device. As shown above, inflation ports may be located near the proximal end of the device which is positioned nearest the cardia in situ. After attachment with the device is assessed, an inflation medium can be introduced and/or extracted, depending on whether the particular structural component is to be enlarged, deflated, or have a buoyancy adjustment. Optionally, an incising instrument could be introduced through the endoscope to penetrate and deflate any filled compartment to reduce the overall volume of the device and improve accommodation of the device.

Additionally, the device can be adapted to allow a patient to detect leakage or impending leakage. In some current balloon embodiments, methylene blue dye or Indian ink, pH-sensitive markers, or any other biocompatible colored agent, can be added to the filling fluid, usually saline, prior to inflation in order to facilitate leakage detection. For example, if the methylene blue leaks into the stomach, a blue color will be present in the patient's excrement.

The wall of the device can be formed using suitable techniques known in the art. For example, an outermost layer and innermost layer can be used. Where there is more than one layer, the layers can be manufactured by either dipping a mold successively into solutions of different materials that dry and cure or preferably by successive precision injections of materials into a mold. Typically, the outermost layer is made of one or more materials, such as silicone rubber, selected primarily for their non-abrasiveness, biocompatibility in the stomach, and resistance to an acidic environment. The innermost layer is then made of materials selected primarily for their resistance to structural fatigue and impermeability to the filling fluid. The inner layer could have biocompatibility of a shorter duration than the outermost layer. The two layers are either bonded together to function as a single wall or left unbonded such that the layers could slide by each other except at certain attachment points. Other structural materials and elements can also be employed without departing from the scope of the invention. For example, the durability may be enhanced by incorporating other structural elements in the layers, such as a mesh made of metal, polymer, or high strength fibers, such as Kevlar.

The present invention further provides a wireless failure detection system for gastric balloons and methods for their deployment and use. The failure detection system can be configured from two probes, a transmitter, and a detector, such as a wireless transmitter and wireless detector. The wireless detector can be adapted to use radio frequency as the signal transmission of choice. However, as will be appreciated by those skilled in the art, it does not exclude other carrier waves, such as light or acoustic, or via physical properties, such as magnetism or temperature. The probes are connected electronically to the wireless transmitter, which can emit a signal recognized by the detector. Upon detection of a change in condition, the detector can be adapted to notify the patient that the integrity of the balloon is compromised and to seek medical assistance. Change in condition can include, for example, change in pH of the contents of a chamber, change in humidity of a chamber, appearance of liquid in a chamber, change in pressure, etc. Alternatively, the device provides for a wireless manual release button or manual override failure system. The device can be configured with a wireless sensor located proximal to the access valve. When pressure changes cause the patient to feel discomfort, the panic button can be triggered on a wireless remote to open the valve to facilitate the release of pressure.

The system can be designed to function in a variety of algorithms to notify the patient in a simple, unequivocal fashion. For example, in a toggle algorithm, the transmitter is either on in the static state or preferably off in order to reduce the need for power. Upon direct contact with the stomach contents, the probe causes the transmitter to turn the signal off or preferably on to be able to send a wireless signal on a continuous basis. The wireless signal or lack thereof is recognized by the detector to notify the patient that the integrity of the balloon is compromised. Alternatively, the algorithm could be based on time, amplitude, frequency, or some other parameter. For example, the transmitter may send a wireless signal at a predetermined time interval in its static state. The detector recognizes the length of the interval as normal and the existence of the signal as the system in working order. Upon direct contact with the stomach contents by the probes, the transmitter is enabled to send the same signal at different time intervals or a different signal, which is recognized by the detector to notify the patient that the integrity of the balloon is compromised. The lack of a signal is recognized by the detector to notify the patient of a detection system malfunction and potential compromise of the integrity of the balloon.

Optionally, more than one probe or more than one type of probe may be placed internally in different parts or components in the device so that the particular part or component which failed may be identified based on which probe was activated. The transmitter would send different signals for the receiver to display the source of the failure. The internal probe could be of any shape and is disposed in the interior or preferably in the wall of the device.

The detection material could be any metal, polymer, fiber, or combination thereof, with or without any coating that can generate an electrical charge or enable flow of electric current when a change in condition occurs.

The transmitter can be a simple wireless signal generator triggered by an electric current or preferably a transponder using the well-established RFID technology, i.e., produces a wireless signal when triggered by an interrogating signal. The electric charge generated or the electric current enabled by the probe in contact with the stomach contents enables the transmitter to emit or causes it to emit a wireless signal. Typically, the transponder is powered by the interrogating radio frequency signal so that no power source of its own is required. Alternatively, the transmitter could be powered by a micro battery or by the electrical power generated by a chemical reaction. For protection from degradation by an acidic and electrolyte solution and become potentially toxic, the transmitter or transponder circuit is encased in a highly resistant material, such as silicon rubber or stainless steel. The transmitter or transponder circuit can be placed on the exterior, embedded in the wall, or preferably in the interior of the balloon for further shielding from chemical degradation and mechanical stress. It can be placed in any orientation, preferably in the plane where the antenna is most sensitive and the transmitter is most effective in sending and receiving signals through body tissue.

The wireless signal from the transmitter is recognized by a detector external to the body. The detector could be simply a receiver tuned to the transmitter's signal or, preferably, a combination of both a transmitter of a signal to interrogate the transponder and a receiver to distinguish the different signals from the transponder. The detector is preferably powered by batteries and portable enough to be worn on a wristband or belt or can be placed conveniently near a place where the patient spends most of his time. Upon receiving a signal that a breach has occurred, the detector will alert the patient to seek medical assistance or alert medical professionals directly through other devices, such as Bluetooth linked to an autodial telephone. The alarm could be auditory, such as beeping sounds, visual, such as flashing LED's or a LCD display, sensory, such as vibrations, or preferably a combination of any or all of the above.

Optionally, the detector could have different auditory, visual, sensory, or different combinations to identify the source of the detected breach, especially with more than one probe or more than one type of probe. For example, LED's of different colors or different sounds could be used. The alarm could further indicate the seriousness of the breach. For example, when multiple probes detect a breach, the volume of the alarm would increase to a higher level.

As a further option, at least a portion of the exterior of the device will be coated or impregnated with an anti-microbial and/or adhesion resistant agent. Preferably, the entire exposed surface of all components of the balloon will be so coated or impregnated to inhibit colonization of the balloon by bacteria or other microbes, and/or reduce possible accumulation of food particles on the device. Suitable anti-microbial agents include polyethylenetetrafluoride (PTFE), and antibiotics.

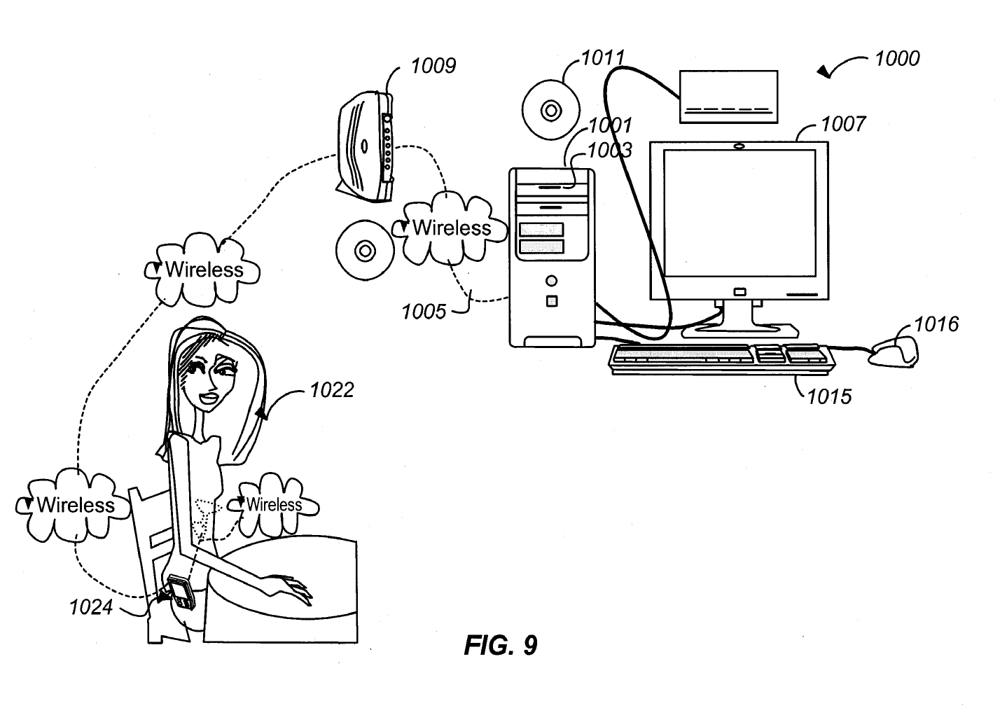

FIG. 9 is a diagram showing a representative example logic device through which reviewing or analyzing data relating to the present invention can be achieved. Such data can be in relation to, for example, pH and pressure. A computer system (or digital device) 1000 that may be understood as a logical apparatus that can read instructions from either media 1011 and/or a network port 1005, which can optionally be connected to server 1009 having fixed media. The computer system 1000 can also be connected to the Internet or an intranet. The system includes CPU 1001, disk drives 1003, optional input devices, illustrated as keyboard 1015 and/or mouse 1016 and optional monitor 1007. Data communication can be achieved through the indicated communication medium to a server 1009 at a local or a remote location. The computer system 1000 can be at a patient's home, or in a remote location, such as a hospital or physician's office. The communication medium can include any means of transmitting and/or receiving data. For example, the communication medium can be a network connection, a wireless connection or an internet connection. It is envisioned that data relating to the present invention can be transmitted over such networks or connections. The computer system can be adapted to communicate with an participant parameter monitor and/or an apparatus on which a participant is engaged in exercise.

A patient 1022 is connected to the system 1000 using a wireless monitoring device 1024 that communicates with an implanted intragastric balloon (e.g., 100-700) to assess the condition of the balloon and/or a patient treatment regimen. The monitoring device can be used to interact with the system. The monitoring device can be a handheld device for use in a point-of-care setting. As will be appreciated by those skilled in the art, the computer system, or digital device, 1000 can be any suitable device.

As discussed above, any of the embodiments of the intragastric device of FIGS. 1-7 include, for example, one or more sensors to assess selected parameters. The sensors may be capable of measuring a biologic function from the participant, measuring a device condition or measuring a change in device environment. Communication from the sensors to the monitoring device could be achieved by any number of mechanisms, as would be appreciated by those skilled in the art. See, for example, US 2005/0135039 entitled Electric Circuit and Transmission Method for Telemetric Transmission (Klemetti), US 2005/0130802 entitled Arrangement, Method and Computer Program for Determining Physical Activity Level of Human Beings (Kinnunen), US 2005/0111307 entitled Electronic Wrist Device (Saaski et al.), US 2005/0111306 entitled Portable Wrist-Worn Personal Electronic Device (Saaski et al.), US 2005/0017850 entitled Mechanical Measuring Device and a Measuring Method (Nissala), US 2005/0004436 entitled Method and Device for Weight Management of Humans (Nissala), US 2004/0220738 entitled Portable Personal Data Processing Device (Nissala), and US 2004/0220485 entitled Method and Device for Measuring Heart Rate, and for Manufacturing the Device (Rytky). U.S. Pat. Nos. 6,832,109 entitled Wrist-Worn Device for Displaying and Setting Heart Rate Parameters (Nissala); 6,754,517 entitled Apparatus for Measuring Electrocardiograph Signal (Nissila); 6,714,812 entitled Method of Performing Operating Settings in Heart Rate Measurement Arrangement, and Heart Rate Measurement Arrangement (Karjalainen); 6,687,535 entitled Controlling of Fitness Exercise (Hautala et al.); 6,605,044 entitled Caloric Exercise Monitor (Bimbaum); 6,584,344 entitled Method and Apparatus for Measuring Heart Rate (Hannula); 6,553,247 entitled Electrode Belt of Heart Rate Monitor (Rytky); 6,540,686 entitled Measurement Relating to Human Body (Heikkila et al.); 6,954,661 entitled Blood Sugar Measuring Apparatus (Cho et al.).

### IV. Methods of Use

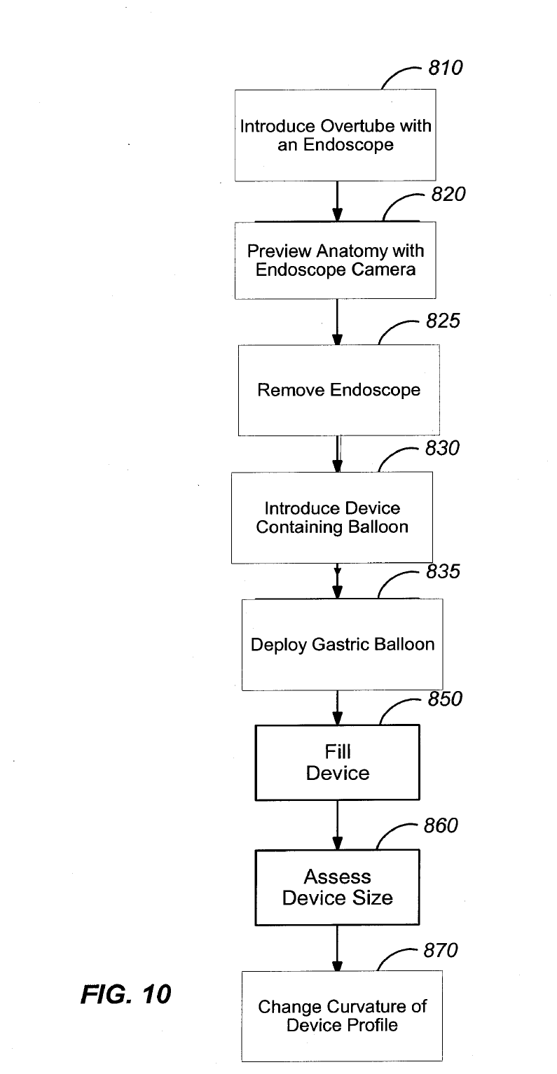

The present invention further provides methods for treating obesity in a patient. The methods comprise introducing a device into the patient's stomach. The device is then filled with an incompressible fluid to provide a fixed support geometry. At least a portion of a separate space-filling compartment is then filled at least partly with a compressible fluid, and/or typically a gas such as air, nitrogen, or the like, within the remainder (if any) being filled with an incompressible material, such as a liquid, gel, slurry, or the like. In this way, the buoyancy of the balloon may be controlled within the limits described above. FIG. 10 illustrates a flow chart of a method for deploying the device. The device is introduced into the stomach through the mouth without the need for surgery 810. The physician may optionally conduct an initial examination of the stomach by inserting an endoscope with an overtube into a patient's mouth and down into the esophagus. The overtube has a dimension known in the art, for example 1.6-2.0 cm in diameter. Thereafter an endoscope is advanced through the overtube 820. If no abnormalities are observed in the esophagus, stomach and duodenum, the physician removes the endoscope 825 and places the device containing a balloon through the overtube 835. The endoscope is reinserted into the mouth via the overtube. The use of an endoscope allows the physician to adjust the placement of the device as desired. Once the device is deployed into the stomach 835, the various chambers of the device are filled with material suitable material to achieve a first profile 850. As will be appreciated by those skilled in the art, a first chamber can be filled with fluid and then a second chamber can be filled with gas, or vice versa. The size of the deployed device can then be assessed to determine whether the device should be larger or smaller 860. Later during the same procedure, or in a subsequent procedure, the profile of the device can be changed 870, either reduced or increased, if necessary. For example, it may be desirable to reduce the profile in order to reduce a patient's feeling of being overly full. Alternatively, it may be desirable to increase the profile if the patient is not experiencing any weight loss, or if the weight loss has plateaud. Thus, the device configuration within the stomach can be changed so that the device has a greater or lesser curvature along its length to either conform more or less to the curvature of the interior of the stomach.

### V. Methods of Treatment

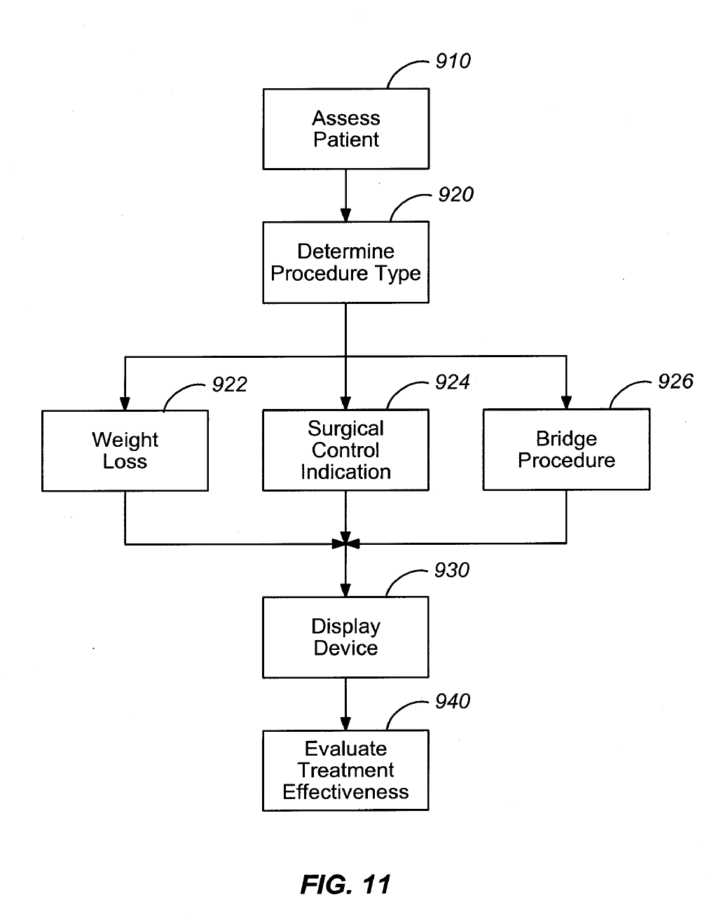

FIG. 11 illustrates a flow chart of a method for treating a patient with the device. The device is indicated for treatment of pediatric obesity, as well as adult weight related issues. In initial physiological and psychological assessment is performed of the patient 910 to determine their suitability for the procedure and the likelihood that the patient will comply with behavioral modification requirements. A determination is made of the type of procedure that the device is intended to achieve 920. For example, the device is suitable for weight loss 922, or use when a patient is contraindicated for surgical intervention 924, or as a bridge procedure for a gastric bypass 926. Once it is determined that the patient is suitable for the procedure, the device is deployed 930. After a period of time, the effectiveness of the treatment is evaluated 940 to determine whether weight loss is occurring and, if so, whether it is occurring at a safe and effective pace (generally considered to be 2 lbs per week). Once the effectiveness of the procedure is determined, the device inflation can be adjusted in situ (either increased or decreased). Thereafter, further evaluations occur at suitable time intervals, e.g., monthly, bimonthly, etc. with further adjustments as required.

A variety of protocols are described for the treatment of obesity. These protocols are provided for illustration purposes.

A. Patient Evaluation

At an initial examination can involve one or more of the following:
The physician takes a complete history and administers a physical examination
The patient is instructed to taper down, and stop NSAIDS (a common cause of peptic ulcer), 1-2 weeks before the gastric balloon placement
A complete evaluation of blood chemistry is performed
Helicobacter Pylori antibody is checked Helicobacter Pylori is a common causes of peptic ulcer)
EKG is performed to exclude cardiac condition
Chest x-ray and/or pulmonary function test is taken to assess pulmonary condition
An upper gastrointestinal series (x-ray) are taken to exclude any abnormalities in the upper gastrointestinal tract, and also evaluate the anatomy and shape of stomach in order to place a right size of balloon for the patient.

At subsequent examinations:
Patient consults a Dietitian to review dietary habits
Patient is evaluated for underlying psychological conditions
Patient engages in weight control program for 3 months to assess patient's suitability for protocol and likelihood of compliance with post-procedure instructions.

If the patient has no contraindications to the upper endoscopy or conscious sedation and patient has lost a prescribed amount of weight during the weight control program, then the upper endoscopy with balloon placement is scheduled at, for example, an outpatient endoscopy center.

B. Device Implantation Procedure

Once the patient has checked into the hospital or an outpatient endoscopy center, and has been prepared for the procedure, one or more of the following steps are performed:
Start conscious sedation
Insert endoscopy with an overtube: an overtube is used to avoid trauma, protect the airway, and because it makes it easier to pass the endoscope and balloon into the stomach
Push the overtube into the esophagus when the endoscope is inside of the esophagus
Complete the upper gastrointestinal examination with the upper endoscope
Pull the scope out but leave the overtube in the esophagus
A pre-packed balloon with a long shaft, and the diameter of the wrapped balloon is less than 1.5 cm. was inserted into the stomach through the overtube.
The endoscope is re-insert into the stomach.
The shaft of the balloon has a channel in the center. The shaft is made from metal, such as steel, or any other suitable biocompatible material. The distal end of the shaft engages the port at the balloon's proximal end and a thin needle or catheter device connecting the shaft is positioned inside the balloon. The proximal end of the shaft outside the human body has a port. The balloon is inflated via the proximal port with the needle by, for example, injecting liquid fist, then air.
When the balloon is inflated at the pre-setting pressure. The shaft is disengaged from the balloon. The shaft and the endoscope are pulled out, followed by the overtube to complete the procedure.

C. Post-Implantation Protocol

Once the device has been implanted a variety of protocol steps can be employed, for example:
Antacid medication is given daily with anti nausea medication as needed.
Educate patient on techniques for monitoring pH and monitoring devices
Nurse will call in 24 hours to check the patient
Physician follow up in 1-2 weeks, with periodic re-checking thereafter

If there are no problems or complications within 3 months, but the patient is not loosing weight, the procedure at Section B can be performed again to replace the currently deployed device with another balloon. For example, the device can be replaced with another device that has a different ratio of liquid and air and different size of balloons (different in the proximal balloon or distal balloon). Alternatively, the deployed device can be left in place and reduced or increased in size, or the ratio of liquid and air can be changed to accommodate the patient.

The balloon should be replace every six months with different mixture of liquid and gas, different size of proximal balloon and distal balloon, or add the third balloon to the proximal balloon.

D. Identification of End of Treatment

The end point for overall treatment can be identified in a variety of ways. For example,
the goal of weight loss is achieved by the patient
the patient developed complications
the patient achieved target initial weight loss before gastric bypass surgery

E. Removal of the Device

In one aspect of the method, the balloon is punctured, e.g. with a special knife like device to deflate the balloon. Thereafter, the port at the proximal balloon for inflation and deflation of the balloon is grasped and snared by a retrieval device, and then pulled out by the endoscope via the overtube.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that any claims that are presented by the inventors define the scope of the invention and that methods and structures within the scope of these claims and their equivalents be covered thereby.

What is claimed is:

1. A device for treatment of obesity in a patient comprising:
an inflatable balloon having a proximal end and a distal end, the inflatable balloon further comprising an interior chamber adapted and configured to receive a filling material;
a port in communication with the interior chamber of the inflatable balloon
wherein the inflatable balloon is adapted and configured to assume a predetermined shape in situ and further
wherein the inflatable balloon is adapted and configured to have a distal diameter less than or equal to a proximal diameter.

2. The device of claim 1 wherein the device has a shape controller along a portion of its length.

3. The device of claim 2 wherein the shape controller is a bar, joint or rod.

4. The device of claim 2 wherein the shape controller is formed from a shape memory material.

5. The device of claim 1 further comprising a plurality of inflatable balloons.

6. The device of claim 1 further comprising two or more prongs extending outwardly from a body of the balloon positioned at the distal end.

7. The device of claim 1 further comprising two or more prongs extending outwardly from a body of the balloon positioned at the proximal end.

8. The device of claim 1 further comprising dimples on an exterior surface of the inflatable balloon.

9. The device of claim 1 further comprising a sensor.

10. The device of claim 9 wherein the sensor is adapted and configured to be positioned on an interior surface of the balloon.

11. The device of claim 9 wherein the sensor is adapted and configured to be positioned on an exterior surface of the balloon.

12. The device of claim 9 wherein the sensor is adapted and configured to detect a change in condition.

13. The device of claim 12 wherein the change in condition is at least one of pH or pressure.

14. The device of claim 9 wherein the sensor is adapted and configured to wirelessly communicate with the patient.

15. The device of claim 9 wherein the sensor is adapted and configured to wirelessly communicate with a patient's healthcare provider.

16. The device of claim 1 further comprising a manual release button wherein the manual release button is adapted and configured to facilitate the release of pressure from the device by the patient.

17. The device of claim 1 wherein the port is adapted and configured to engage a retrieval device.

18. The device of claim 1 wherein the device further comprises a proximal section, a distal section and an intermediate section, further wherein the diameter of the proximal section does not equal the diameter of the distal section and does not equal the diameter of the intermediate section.

19. The device of claim 1 wherein the port is further adapted and configured to communicate with a plurality of chambers.

20. The device of claim 1 wherein the device further comprises a plurality of ports and a plurality of chambers, the plurality of ports adapted and configured to communicate the plurality of chambers.

21. A device for treatment of obesity in a patient comprising:
an inflatable balloon having a proximal end and a distal end, the inflatable balloon further comprising an interior chamber adapted and configured to receive a filling material;
a port in communication with the interior chamber of the inflatable balloon
wherein the inflatable balloon is adapted and configured to achieve a deployment shape at least partially conformable to an interior dimensional shape of a stomach of the patient and further wherein the inflatable balloon is adapted and configured to have a distal diameter less than or equal to a proximal diameter.

22. The device of claim 21 wherein the device has a shape controller along a portion of its length.

23. The device of claim 22 wherein the shape controller is a bar, joint or rod.

24. The device of claim 22 wherein the shape controller is formed from a shape memory material.

25. The device of claim 21 further comprising a plurality of inflatable balloons.

26. The device of claim 21 further comprising two or more prongs extending outwardly from a body of the balloon positioned at the distal end.

27. The device of claim 21 further comprising two or more prongs extending outwardly from a body of the balloon positioned at the proximal end.

28. The device of claim 21 further comprising dimples on an exterior surface of the inflatable balloon.

29. The device of claim 21 further comprising a sensor.

30. The device of claim 21 wherein the sensor is adapted and configured to be positioned on an interior surface of the balloon.

31. The device of claim 30 wherein the sensor is adapted and configured to be positioned on an exterior surface of the balloon.

32. The device of claim 30 wherein the sensor is adapted and configured to detect a change in condition.

33. The device of claim 32 wherein the change in condition is at least one of pH or pressure.

34. The device of claim 30 wherein the sensor is adapted and configured to wirelessly communicate with the patient.

35. The device of claim 30 wherein the sensor is adapted and configured to wirelessly communicate with a patient's healthcare provider.

36. The device of claim 21 wherein the port is adapted and configured to engage a retrieval device.

37. The device of claim 21 further comprising a manual release button wherein the manual release button is adapted and configured to facilitate the release of pressure from the device by the patient.

38. The device of claim 21 wherein the device further comprises a proximal section, a distal section and an intermediate section, further wherein the diameter of the proximal section does not equal the diameter of the distal section and does not equal the diameter of the intermediate section.

39. The device of claim 21 wherein the port is further adapted and configured to communicate with a plurality of chambers.

40. The device of claim 21 wherein the device further comprises a plurality of ports and a plurality of chambers, the plurality of ports adapted and configured to communicate with the plurality of chambers.

41. A method for treating obesity in a patient, the method comprising:
introducing a device comprising an inflatable balloon having a proximal end and a distal end, the inflatable balloon further comprising an interior chamber, and a port in communication with the interior chamber of the inflatable balloon wherein the inflatable balloon is adapted and configured to achieve a deployed shape at least partially conformable to an interior dimensional shape of a stomach of the patient;
expanding the balloon to provide a conformable geometry; and
at least partly filling the chamber of the balloon with a compressible and/or incompressible fluid.

42. The method of claim 41, further comprising the steps of determining a size of the stomach and selecting a balloon of a proper size.

43. The method of claim 42, wherein the step of determining further comprises visually examining the gastric cavity of the stomach through a gastroscope, externally scanning with X-rays, or externally scanning with ultrasound.

44. The method of claim 42, wherein the size is determined while the stomach is filled with a biocompatible medium.

45. The method of claim 44, wherein the balloon size is selected to leave an unobstructed stomach volume surrounding the sides of the device in the range from 10 cm³ to 100 cm³ after the balloon is inflated.

46. The method of claim 41, wherein the introducing step further comprises passing the device in a deflated configuration into the stomach through an endoscope.

47. The method of claim 41 wherein each chamber is inflated through the port with an inflation tube removably attached to the balloon.

48. The method of claim 47, wherein the device has at least two interior chambers and further wherein the at least two chambers are inflated by the inflation tube.

49. The method of claim 41, wherein the chambers are filled with a mixture of compressible and incompressible fluid to control the buoyancy of the gastric balloon in the stomach.

50. The method of claim 49, wherein the mixture of compressible and incompressible fluids is selected to provide a generally neutral buoyancy in the stomach.

51. The method of claim 41, further comprising the step of deflating the chambers and removing the deflated balloon from the stomach.

52. The method of claim 51, wherein the step of deflating further comprises breaching one or more walls of each chamber.

53. The method of claim 41, further comprising the step of adjusting a fill volume of at least one chamber after filling has been completed.

54. The method of claim 53 wherein the step of adjusting the fill volume further comprises reattaching an inflation tube to one or more space-filling compartments, and filling or removing inflation fluid through the reattached inflation tube.

55. A method for deploying a gastric balloon in a patient, the method comprising the steps of:
introducing the gastric balloon into a stomach of the patient; and
separately inflating a plurality of isolated chambers within the balloon, wherein the chambers have individual volumes such that the collective volume of the chambers remaining inflated after the deflation of any single chamber is such that the balloon is prevented from passing through the pylorus.

56. The method of claim 55, further comprising the step of detecting a substance which is released into the stomach by a partially or fully ruptured balloon chamber wherein the detection is achieved by detecting excretion, secretion, exhalation, or regurgitation by the patient.

57. A method for selecting a gastric balloon for a patient, the method comprising the steps of:
determining an internal volume of a stomach of the patient while the stomach is filled with a biocompatible filling medium; and
selecting a balloon having a filling volume less than the determined volume by a preselected amount.

58. The method of claim 57, wherein the preselected amount is in the range from 10 cm³ to 100 cm³.

59. The method of claim 58, wherein the biocompatible filling medium comprises liquid or solid food.

60. The method of claim 58, wherein the internal volume is measured by external X-ray, external ultrasound, or by internal endoscopic measurement.

61. A method of in vivo monitoring a condition of a gastric balloon comprising:
introducing a device comprising an inflatable balloon having a proximal end and a distal end, the inflatable balloon further comprising an interior chamber, a port in communication with the interior chamber of the inflatable balloon wherein the inflatable balloon is adapted and configured to achieve a deployed shape at least partially conformable to an interior dimensional shape of a stomach of the patient, and a sensor adapted and configured to sense a condition; and
sensing a condition of the device.

62. The method of claim 61 further comprising the step of performing a plurality of calibrations of the sensor.

63. The method of claim 61 wherein the step of sensing further comprises sensing a physiologic condition of a content of the stomach.

64. The method of claim 63 further comprising the step of instructing a patient to take an antacid.

65. The method of claim 63 further comprising the step of advising a healthcare provider of the results of the sensing step.

66. The method of claim 61 wherein the step of sensing further comprises sensing a condition of the device.

67. The method of claim 66 further comprising the step of alerting a patient of the condition of the device.

68. The method of claim 66 further comprising the step of instructing a patient to contact a healthcare provider.

69. The method of claim 66 further comprising alerting a healthcare provide of the condition of the device.

70. A wireless device for treatment of obesity in a patient comprising:
an inflatable balloon having a proximal end and a distal end, the inflatable balloon further comprising an interior chamber adapted and configured to receive a filling material;
a sensor connected to the inflatable balloon adapted and configured to sense a parameter; and
a port in communication with the interior chamber of the inflatable balloon.

71. The device of claim 70 wherein the device has a shape controller along a portion of its length.

72. The device of claim 71 wherein the shape controller is a bar, joint or rod.

73. The device of claim 71 wherein the shape controller is formed from a shape memory material.

74. The device of claim 70 further comprising a plurality of inflatable balloons.

75. The device of claim 70 further comprising two or more prongs extending outwardly from a body of the balloon positioned at the distal end.

76. The device of claim 70 further comprising two or more prongs extending outwardly from a body of the balloon positioned at the proximal end.

77. The device of claim 70 further comprising dimples on an exterior surface of the inflatable balloon.

78. The device of claim 70 wherein the sensor is adapted and configured to be positioned on an interior surface of the balloon.

79. The device of claim 70 wherein the sensor is adapted and configured to be positioned on an exterior surface of the balloon.

80. The device of claim 70 wherein the sensor is adapted and configured to detect a change in condition.

81. The device of claim 80 wherein the change in condition is at least one of pH or pressure.

82. The device of claim 70 wherein the sensor is adapted and configured to wirelessly communicate with the patient.

83. The device of claim 70 wherein the sensor is adapted and configured to wirelessly communicate with a patient's healthcare provider.

84. The device of claim 70 wherein the port is adapted and configured to engage a retrieval device.

85. The device of claim 70 wherein the device further comprises a proximal section, a distal section and an intermediate section, further wherein the diameter of the proximal section does not equal the diameter of the distal section and does not equal the diameter of the intermediate section.

86. The device of claim 70 wherein the port is further adapted and configured to communicate with a plurality of chambers.

87. The device of claim 70 wherein the device further comprises a plurality of ports and a plurality of chambers, the plurality of ports adapted and configured to communicate the plurality of chambers.

88. A kit for treatment of obesity in a patient comprising:
a device comprising an inflatable balloon having a proximal end and a distal end, the inflatable balloon further comprising an interior chamber adapted and configured to receive a filling material; a port in communication with the interior chamber of the inflatable balloon; wherein the inflatable balloon is adapted and configured to assume a predetermined shape in situ and further wherein the inflatable balloon is adapted and configured to have a distal diameter less than or equal to a proximal diameter;
one or more delivery materials; and
a retrieval device.

89. A kit for treatment of obesity in a patient comprising:
a device comprising an inflatable balloon having a proximal end and a distal end, the inflatable balloon further comprising an interior chamber adapted and configured to receive a filling material; a port in communication with the interior chamber of the inflatable balloon, wherein the inflatable balloon is adapted and configured to achieve a deployment shape at least partially conformable to an interior dimensional shape of a stomach of the patient and further wherein the inflatable balloon is adapted and configured to have a distal diameter less than or equal to a proximal diameter;
one or more delivery materials; and
a retrieval device.

90. A kit for treatment of obesity in a patient comprising:
a device comprising an inflatable balloon having a proximal end and a distal end, the inflatable balloon further comprising an interior chamber adapted and configured to receive a filling material; a sensor connected to the inflatable balloon adapted and configured to sense a parameter; and a port in communication with the interior chamber of the inflatable balloon;
one or more delivery materials; and
a retrieval device.

91. An intragastric balloon device comprising:
a hollow pliable sac defining an interior space, the sac further comprising a proximal end and a distal end and an exterior surface;
at least one protrusion extending from the exterior surface of the sac; and
an access port adapted to provide material access to the interior space.

92. The device of claim 89 further comprising at least one prong extending from the exterior surface of the sac at the distal end.

93. The device of claim 89 wherein the interior space further comprises at least two isolated chambers adapted and configured to be filled with a material.

94. The device of claim 93 wherein the access port is configured to provide material access to the at least one isolated chamber.

95. The device of claim 89 further comprising a second access port.

96. The device of claim 95 wherein a first access port provides material access to a first chamber in the interior space and the second access port provides material access to a second chamber in the interior space.

97. The device of claim 89 wherein the distal end of the device has a smaller diameter than the proximal end of the device.

98. The device of claim 89 further comprising at least one prong extending from the exterior surface of the sac at the proximal end.

99. The device of claim 89 further comprising a sensor.

100. The device of claim 99 wherein the sensor is adapted and configured to be positioned on the exterior surface of the sac.

101. The device of claim 99 wherein the sensor is adapted and configured to communicate wirelessly with a data receiver.

102. The device of claim 99 wherein the sensor is adapted and configured to detect a change in condition.

103. The device of claim 102 wherein the change in condition is at least one of pH or pressure.

104. The device of claim 102 wherein the diameter of the sac varies with the length of the sac.

105. The device of claim 104 wherein the diameter of the sac at the proximal end is greater than the diameter of the sac at the distal end.

106. The device of claim 105 further comprising a shape controller.

107. The device of claim 106 wherein the shape controller is a bar, joint or rod.

108. The device of claim 106 wherein the shape controller is formed from a shape memory material.

109. An intragastric balloon device for positioning in a stomach of a patient comprising
a hollow pliable sac defining an interior space, the sac further comprising a proximal end and a distal end and an exterior surface;
a series of protrusions extending from the exterior surface of the sac;
an access port adapted to provide material access to the interior space; and
a series of prongs located on the distal end of the sac, wherein the prongs are adapted to prevent migration of the sac.

110. The device of claim 109 wherein the interior space further comprises at least two isolated chambers adapted and configured to be filled with a material.

111. The device of claim 109 wherein the access port is configured to provide material access to the at least one isolated chamber.

112. The device of claim 109 further comprising a second access port.

113. The device of claim 112 wherein a first access port provides material access to a first chamber in the interior space and the second access port provides material access to a second chamber in the interior space.

114. The device of claim 109 wherein the distal end of the device has a smaller diameter than the proximal end of the device.

115. The device of claim 109 further comprising at least one prong extending from the exterior surface of the sac at the proximal end.

116. The device of claim 109 further comprising a sensor.

117. The device of claim 116 wherein the sensor is adapted and configured to be positioned on the exterior surface of the sac.

118. The device of claim 117 wherein the sensor is adapted and configured to communicate wirelessly with a data receiver.

119. The device of claim 116 wherein the sensor is adapted and configured to detect a change in condition.

120. The device of claim 119 wherein the change in condition is at least one of pH or pressure.

121. The device of claim 119 wherein the diameter of the sac varies with the length of the sac.

122. The device of claim 109 wherein the diameter of the sac varies with the length of the sac.

123. The device of claim 122 wherein the diameter of the sac at the proximal end is greater than the diameter of the sac at the distal end.

124. The device of claim 123 further comprising a shape controller.

125. The device of claim 124 wherein the shape controller is a bar, joint or rod.

126. The device of claim 124 wherein the shape controller is formed from a shape memory material.

127. An intragastric balloon device comprising
a hollow pliable sac, the sac further comprising a proximal end and a distal end and an exterior surface;
an interior space defined within the sac wherein the interior space further comprises at least two isolated chambers wherein the isolated chambers are adapted to be filled with a media; and
a controlling mechanism adapted to conform and maintain the device in a user defined configuration.

128. The device of claim 127 further comprising at least one prong extending from the exterior surface of the sac at the distal end.

129. The device of claim 127 further comprising an access port wherein the access port is adapted and configured to provide material access to the at least one isolated chamber.

130. The device of claim 127 further comprising a second access port.

131. The device of claim 130 wherein a first access port provides material access to a first chamber in the interior space and the second access port provides material access to a second chamber in the interior space.

132. The device of claim 127 wherein the distal end of the device has a smaller diameter than the proximal end of the device.

133. The device of claim 127 further comprising at least one prong extending from the exterior surface of the sac at the proximal end.

134. The device of claim 127 further comprising a sensor.

135. The device of claim 134 wherein the sensor is adapted and configured to detect a change in condition.

136. The device of claim 135 wherein the change in condition is at least one of pH or pressure.

137. The device of claim 134 wherein the sensor is adapted and configured to be positioned on the exterior surface of the sac.

138. The device of claim 134 wherein the sensor is adapted and configured to communicate wirelessly with a data receiver.

139. The device of claim 127 wherein the diameter of the sac varies with the length of the sac.

140. The device of claim 139 wherein the diameter of the sac at the proximal end is greater than the diameter of the sac at the distal end.

141. The device of claim 127 wherein the controlling mechanism is a bar, joint or rod.

142. The device of claim 127 wherein the controlling mechanism is formed from a shape memory material.

143. An intragastric balloon device comprising:
a first inflatable chamber;
a second inflatable chamber; and
an elongate neck region connecting the first and second inflatable chambers wherein the neck region is adapted and configured to conform and maintain the device in a user defined configuration.

144. The device of claim 143 further comprising at least one prong extending from the exterior surface of the sac at the distal end.

145. The device of claim 143 further comprising an access port wherein the access port is configured to provide material access to the at least one chamber.

146. The device of claim 143 further comprising a second access port.

147. The device of claim 146 wherein a first access port provides material access to a first chamber in the interior space and the second access port provides material access to a second chamber in the interior space.

148. The device of claim 143 wherein the first inflatable chamber of the device has a smaller diameter than the second inflatable chamber of the device.

149. The device of claim 143 further comprising at least one prong extending from the second inflatable chamber wherein the second inflatable chamber is located proximal to the pylorus.

150. The device of claim 143 further comprising a sensor.

151. The device of claim 150 wherein the sensor is adapted and configured to detect a change in condition.

152. The device of claim 151 wherein the change in condition is at least one of pH or pressure.

153. The device of claim 150 wherein the sensor is adapted and configured to be positioned on the exterior surface of the sac.

154. The device of claim 150 wherein the sensor is adapted and configured to communicate wirelessly with a data receiver.

155. The device of claim 143 wherein the elongate neck region is a bar, joint or rod.

156. The device of claim 143 wherein the elongate neck region is formed from a shape memory material.
